(19) United States
(12) Patent Application Publication
(10) Pub. No.: US 2003/0104426 A1
(43) Pub. Date: Jun. 5, 2003

(54) Signature genes in chronic myelogenous leukemia

(76) Inventors: Peter S. Linsley, Seattle, WA (US); Mao Mao, Kirkland, WA (US); Hongyue Dai, Bothell, WA (US); Yudong He, Kirkland, WA (US); Jerald Patrick Radich, Sammamish, WA (US)

Correspondence Address: PENNIE AND EDMONDS, 1155 AVENUE OF THE AMERICAS, NEW YORK, NY, 100362711

(21) Appl. No.: 10/171,581

(22) Filed: Jun. 14, 2002

Related U.S. Application Data
(60) Provisional application No. 60/298,914, filed on Jun. 18, 2001.

(51) Int. Cl. C12Q 1/68; G06F 19/00; G01N 33/48; G01N 33/50

(52) U.S. Cl. 435/6; 702/20

(57) ABSTRACT

The present invention relates to genetic markers whose expression is correlated with progression of CML. Specifically, the invention provides sets of markers whose expression patterns can be used to differentiate chronic phase individuals from those in blast crisis. The invention relates to methods of using these markers to distinguish these conditions. The invention also relates to kits containing ready-to-use microarrays and computer software for data analysis using the statistical methods disclosed herein.

This application claims benefit of U.S. Provisional Application No. 60/298,914, filed Jun. 18, 2001, which is incorporated by reference herein in its entirety.

This application includes a Sequence Listing submitted on compact disc, recorded on two compact discs, including one duplicate, containing Filename 9301157999.txt, of size 999,424 bytes, created Jun. 12, 2002. The sequence listing on the compact discs is incorporated by reference herein in its entirety.

### 1. FIELD OF THE INVENTION

The present invention relates to the identification of expression changes that occur in the evolution from the chronic phase to blast crisis of chronic myeloid leukemia (CML).

### 2. BACKGROUND OF THE INVENTION

Chronic myeloid leukemia (CML) is a clonal disease that acquires genetic change in a pluripotential hematopoietic stem cell. The altered stem cell proliferates and generates a population of differentiated cells that gradually replaces normal hematopoiesis and leads to a greatly expanded total myeloid mass. One important landmark in the study of CML was the discovery of the Philadelphia (Ph) chromosome in 1960; another was the characterization in 1986 of the BCR-ABL chimeric gene. Until the 1980s, CML was assumed to be incurable. Palliative treatments included radiotherapy and, more recently, alkylating agents, notably busulphan. It has become apparent in the last 20 years that CML can be cured by bone marrow transplantation (BMT), but the proportion of patients eligible for BMT is still relatively small.

The incidence of CML appears to be constant worldwide. It occurs in about 1.0 to 1.5 per 100,000 of the population in all countries where statistics are adequate. CML is a biphasic or triphasic disease that is usually diagnosed in the initial ‘chronic’ or stable phase. The chronic phase lasts typically for 2-7 years. In about 50% patients, the chronic phase transforms unpredictably and abruptly to a more aggressive phase, blast crisis. In the other half of patients, the disease evolves somewhat more gradually, through an intermediate phase described as “accelerated” disease, which may last for months, before transformation to blast crisis. The duration of survival after the onset of transformation is usually only 2-6 months.

In clinical practice, accurate determination of the different phases of CML is important because treatment options, prognosis, and the likelihood of therapeutic response all vary broadly depending on the determination. To date, no set of marker genes that can be used to distinguish chronic phase and blast crisis of CML.

### 3. SUMMARY OF THE INVENTION

The invention provides gene marker sets that distinguish chronic phase CML from blast crisis CML, and methods of use therefor. In one embodiment, the invention provides a method for classifying a cell sample as blast crisis or chronic phase CML comprising detecting a difference in the expression of a first plurality of genes relative to a control, said first plurality of genes consisting of at least 5 of the genes corresponding to the markers listed in Table 1. In specific embodiments, said plurality of genes consists of at least 50, 100, 200, or 300 of the gene markers listed in Table 1. In another specific embodiment, said control comprises nucleic acids derived from a pool of samples from individual chronic phase patients.

The invention further provides a method for classifying a sample as chronic phase or blast crisis by calculating the similarity between the expression of at least 5 of the markers listed in Table 1 in the sample to the expression of the same markers in an chronic phase nucleic acid pool and an blast phase nucleic acid pool, comprising the steps of: (a) labeling nucleic acids derived from a sample, with a first fluorophore to obtain a first pool of fluorophore-labeled nucleic acids; (b) labeling with a second fluorophore a first pool of nucleic acids derived from two or more chronic phase samples, and a second pool of nucleic acids derived from two or more blast phase samples; (c) contacting said first fluorophore-labeled nucleic acid and said first pool of second fluorophore-labeled nucleic acid with said first microarray under conditions such that hybridization can occur, and contacting said first fluorophore-labeled nucleic acid and said second pool of second fluorophore-labeled nucleic acid with said second microarray under conditions such that hybridization can occur, detecting at each of a plurality of discrete loci on the first microarray a first flourescent emission signal from said first fluorophore-labeled nucleic acid and a second fluorescent emission signal from said first pool of second fluorophore-labeled genetic matter that is bound to said first microarray under said conditions, and detecting at each of the marker loci on said second microarray said first fluorescent emission signal from said first fluorophore-labeled nucleic acid and a third fluorescent emission signal from said second pool of second fluorophore-labeled nucleic acid; (d) determining the similarity of the sample to the blast crisis and chronic phase pools by comparing said first fluorescence emission signals and said second fluorescence emission signals, and said first emission signals and said third fluorescence emission signals; and (e) classifying the sample as chronic phase where the first fluorescence emission signals are more similar to said second fluorescence emission signals than to said third fluorescent emission signals, and classifying the sample as blast crisis where the first fluorescence emission signals are more similar to said third fluorescence emission signals than to said second fluorescent emission signals, wherein said first microarray and said second microarray are similar to each other, exact replicas of each other, or are identical, and wherein said similarity is defined by a statistical method such that the cell sample and control are similar where the p value of the similarity is less than 0.01. In a specific embodiment, said similarity is calculated by determining a first sum of the differences of expression levels for each marker between said first fluorophore-labeled nucleic acid and said first pool of second fluorophore-labeled nucleic acid, and a second sum of the differences of expression levels for each marker between said first fluorophore-labeled nucleic acid and said second pool of second fluorophore-labeled nucleic acid, wherein if said first sum is greater than said second sum, the sample is classified as blast crisis, and if said second sum is greater than said first sum, the sample is classified as chronic phase. In another specific embodiment, said similarity is calculated by computing a first classifier parameter P₁ between an chronic phase template and the expression of said markers in said sample, and a second classifier parameter P₂ between an blast crisis template and the expression of said markers in said sample, wherein said P₁ and P₂are calculated according to the formula:

P₁=(\vec{z}ᵢ•\vec{y})/(∥\vec{z}ᵢ∥·∥\vec{y}∥),

wherein \vec{z}₁ and \vec{z}₂ are blast crisis and chronic phase templates, respectively, and are calculated by averaging said second fluorescence emission signal for each of said markers in said first pool of second fluorophore-labeled nucleic acid and said third fluorescence emission signal for each of said markers in said second pool of second fluorophore-labeled nucleic acid, respectively, and wherein \vec{y} is said first fluorescence emission signal of each of said markers in the sample to be classified as chronic phase or blast crisis, wherein the expression of the markers in the sample is similar to blast crisis if P₁<P₂, and similar to chronic phase if P₁>P₂.

The invention further provides a method for identifying marker genes associated with a particular phenotype. In one embodiment, the invention provides a method for determining a set of marker genes whose expression is associated with a particular phenotype, comprising the steps of: (a) selecting the phenotype having two or more phenotype categories; (b) identifying a plurality of genes wherein the expression of said genes is correlated or anticorrelated with one of the phenotype categories, and wherein the correlation coefficient for each gene is calculated according to the equation

ρ=(\vec{c}•\vec{r})/(∥\vec{c}∥·∥\vec{r}∥),

wherein \vec{c} is a number representing said phenotype category and \vec{r} is the logarithmic expression ratio across all the samples for each individual gene, wherein if the correlation coefficient has an absolute value of 0.3 or greater, said expression of said gene is associated with the phenotype category, wherein said plurality of genes is a set of marker genes whose expression is associated with a particular phenotype. In a specific embodiment, said set of marker genes is validated by: (a) using a statistical method to randomize the association between said marker genes and said phenotype category, thereby creating a control correlation coefficient for each marker gene; (b) repeating step (a) one hundred or more times to develop a frequency distribution of said control correlation coefficients for each marker gene; (c) determining the number of marker genes having a control correlation coefficient of 0.3 or above, thereby creating a control marker gene set; and (d) comparing the number of control marker genes so identified to the number of marker genes, wherein if the p value of the difference between the number of marker genes and the number of control genes is less than 0.01, said set of marker genes is validated. In another specific embodiment, said set of marker genes is optimized by the method comprising: (a) rank-ordering the genes by amplitude of correlation or by significance of the correlation coefficients, and (b) selecting an arbitrary number of marker genes from the top of the rank-ordered list.

The invention further provides microarrays comprising the disclosed marker sets. In one embodiment, the invention provides a microarray for distinguishing chronic phase and blast crisis cell samples comprising a positionally-addressable array of polynucleotide probes bound to a support, said polynucleotide probes comprising a plurality of polynucleotide probes of different nucleotide sequences, each of said different nucleotide sequences comprising a sequence complementary and hybridizable to a plurality of genes, said plurality consisting of at least 5 of the genes corresponding to the markers listed in Table 1. The invention further provides for microarrays comprising at least 20, 50, 100, 200, or 300 of the marker genes listed in Table 1.

The invention further provides a kit for determining the CML status of a sample, comprising at least two microarrays each comprising at least 20 of the markers listed in Table 1, and a computer system for determining the similarity of the level of nucleic acid derived from the markers listed in Table 1 in a sample to that in a blast crisis pool and a chronic phase pool, the computer system comprising a processor, and a memory encoding one or more programs coupled to the processor, wherein the one or more programs cause the processor to perform a method comprising computing the aggregate differences in expression of each marker between the sample and blast crisis pool and the aggregate differences in expression of each marker between the sample and chronic phase pool, or a method comprising determining the correlation of expression of the markers in the sample to the expression in the blast crisis and chronic phase pools, said correlation calculated according to Equation (3).

### 4. BRIEF DESCRIPTION OF THE FIGURES

FIG. 1 Experimental procedures for measuring differential changes in mRNA transcript abundance in bone marrow cells used in this study. In each experiment, Cy5-labeled cRNA from one sample X is hybridized on a 25 k human chip together with Cy3-labeled cRNA pool made of cRNA samples from samples 1, 2, . . . N. The digital expression data were obtained by scanning and image processing. The error modeling allowed assignment of a p-value to each transcript ratio measurement.

FIG. 2 Two-dimensional clustering analysis results of 20 samples and 245 significant genes. Clustering of CML patients reveals expression patterns that are predictive of progression to blast crisis. Color represents the log ratio of the gene expression regulation.

FIG. 3 Procedures used in identifying the optimal set of discriminating genes for the purpose of monitoring the disease progression of CML patients.

FIG. 4 t-values and average log ratio for the chronic phase group (type 1) and the blast crisis group (type 2) respectively are shown for each gene. The gene index is sorted by the amplitude of t-values. Genes on the two ends of the list likely contain information about the disease progression.

FIG. 5A T-values for each gene that survived the selection criteria.

FIG. 5B Average log ratio for the chronic phase group (type 1) and the blast crisis group (type 2) respectively. The systematic difference between these two groups over the set of 366 discriminating genes allows the classification of the two groups based on gene expression patterns.

FIG. 6 The expression patterns found in the training data. Displayed in the map is the log ratio for the chronic phase group (upper part)) and the blast crisis group (lower part) respectively. The systematic difference between these two groups over this set of discriminating genes allows the classification of the two groups based on gene expression patterns.

FIG. 7 Similarity measures of each sample to the chronic phase group (Parameter 1) and to the blast crisis group (Parameter 2). Solid symbols are for training data. Open symbols are for predictions.

FIG. 8 Histogram of discriminating parameter for all samples used in training (A) and for all independent samples (B).

FIG. 9 The progression status of all bone marrow samples classified based on the gene expression patterns of 366 discriminating marker genes. Clinical information is listed to the right.

FIG. 10 The progression status of all bone marrow samples classified by support vector machine based on the gene expression patterns of 366 discriminating marker genes.

### 5. DETAILED DESCRIPTION OF THE INVENTION

### 5.1 Introduction

The invention relates to newly-discovered correlations between the expression of certain markers and chronic myclogenous leukemia (CML). A set of genetic markers has been determined, the expression of which correlates with the existence of CML. More specifically, the invention provides for set of genetic markers that can distinguish chronic phase from blast phase Methods are provided for use of these markers to distinguish between these patient groups, and to determine general courses of treatment. Microchip oligonucleotide arrays comprising these markers are also provided, as well as methods of constructing such microarrays.

### 5.2 Definitions

As used herein, “Marker-derived polynucleotides” means the RNA transcribed from a marker gene, any cDNA or cRNA produced therefrom, and any nucleic acid derived therefrom, such as synthetic nucleic acid having a sequence derived from the gene corresponding to the marker gene.

### 5.3 Markers Useful in Diagnosis Progression of CML

### 5.3.1 Marker Sets

The invention provides a set of 366 genetic markers correlated with the existence of CML by clustering analysis. A subset of these markers identified as useful for diagnosis of CML progression is listed in Table 1 (SEQ ID NOS: 1-366). The invention also provides a method of using these markers to distinguish chronic phase from blast phase samples.

TABLE 1
366 gene markers that distinguish blast phase from
chronic stage CML.

| | |
|---|---|
| X15414 | SEQ ID NO 1 |
| U89436 | SEQ ID NO 2 |
| D87459 | SEQ ID NO 3 |
| Y10275 | SEQ ID NO 4 |
| AF027299 | SEQ ID NO 5 |
| M34079 | SEQ ID NO 6 |
| AF054840 | SEQ ID NO 7 |
| Al671741 | SEQ ID NO 8 |
| M72709 | SEQ ID NO 9 |
| D38549 | SEQ ID NO 10 |
| T99512 | SEQ ID NO 11 |
| Y00433 | SEQ ID NO 12 |
| L31801 | SEQ ID NO 13 |
| AF043045 | SEQ ID NO 14 |
| X75252 | SEQ ID NO 15 |
| X53793 | SEQ ID NO 16 |
| M14505 | SEQ ID NO 17 |
| Al557064 | SEQ ID NO 18 |
| J04794 | SEQ ID NO 19 |
| M24194 | SEQ ID NO 20 |
| X17620 | SEQ ID NO 21 |
| X73460 | SEQ ID NO 22 |
| X92720 | SEQ ID NO 23 |
| M58458 | SEQ ID NO 24 |
| Al358246 | SEQ ID NO 25 |
| X76538 | SEQ ID NO 26 |
| Y12065 | SEQ ID NO 27 |
| U28946 | SEQ ID NO 28 |
| H23562 | SEQ ID NO 29 |
| X67951 | SEQ ID NO 30 |
| X62744 | SEQ ID NO 31 |
| M36981 | SEQ ID NO 32 |
| N30076 | SEQ ID NO 33 |
| D45248 | SEQ ID NO 34 |
| AA448663 | SEQ ID NO 35 |
| AB015907 | SEQ ID NO 36 |
| X06994 | SEQ ID NO 37 |
| AA987540 | SEQ ID NO 38 |
| X85545 | SEQ ID NO 39 |
| J04031 | SEQ ID NO 40 |
| AA142859 | SEQ ID NO 41 |
| U20536 | SEQ ID NO 42 |
| X95632 | SEQ ID NO 43 |
| AB007917 | SEQ ID NO 44 |
| D21851 | SEQ ID NO 45 |
| M31523 | SEQ ID NO 46 |
| X02994 | SEQ ID NO 47 |
| J03592 | SEQ ID NO 48 |
| D21262 | SEQ ID NO 49 |
| AF070735 | SEQ ID NO 50 |
| U54778 | SEQ ID NO 51 |
| AF030424 | SEQ ID NO 52 |
| M94065 | SEQ ID NO 53 |
| X52142 | SEQ ID NO 54 |
| M69039 | SEQ ID NO 55 |
| X74801 | SEQ ID NO 56 |
| D43948 | SEQ ID NO 57 |
| M23619 | SEQ ID NO 58 |
| AJ223948 | SEQ ID NO 59 |
| A1214598 | SEQ ID NO 60 |
| J04991 | SEQ ID NO 61 |
| AL691084 | SEQ ID NO 62 |
| AB011124 | SEQ ID NO 63 |
| AA669106 | SEQ ID NO 64 |
| U09086 | SEQ ID NO 65 |
| AL535884 | SEQ ID NO 66 |
| D42054 | SEQ ID NO 67 |
| N32858 | SEQ ID NO 68 |
| S43127 | SEQ ID NO 69 |
| AB020637 | SEQ ID NO 70 |
| AF029893 | SEQ ID NO 71 |
| U43374 | SEQ ID NO 72 |
| AL472106 | SEQ ID NO 73 |
| D42043 | SEQ ID NO 74 |
| M34181 | SEQ ID NO 75 |
| X06323 | SEQ ID NO 76 |
| AJ006291 | SEQ ID NO 77 |
| U03911 | SEQ ID NO 78 |
| Al374994 | SEQ ID NO 79 |
| D84276 | SEQ ID NO 80 |
| X70683 | SEQ ID NO 81 |
| AB014540 | SEQ ID NO 82 |
| AB002330 | SEQ ID NO 83 |
| U32519 | SEQ ID NO 84 |
| D86956 | SEQ ID NO 85 |
| AF001601 | SEQ ID NO 86 |
| Al379662 | SEQ ID NO 87 |
| Al669720 | SEQ ID NO 88 |
| AA142949 | SEQ ID NO 89 |
| U43185 | SEQ ID NO 90 |
| AF008442 | SEQ ID NO 91 |
| Al275895 | SEQ ID NO 92 |
| D90224 | SEQ ID NO 93 |
| U59919 | SEQ ID NO 94 |
| M94856 | SEQ ID NO 95 |
| M83822 | SEQ ID NO 96 |
| X74330 | SEQ ID NO 97 |
| M32578 | SEQ ID NO 98 |
| F040105 | SEQ ID NO 99 |
| U53003 | SEQ ID NO 100 |
| Al253387 | SEQ ID NO 101 |
| Z11692 | SEQ ID NO 102 |
| S73885 | SEQ ID NO 103 |
| X07696 | SEQ ID NO 104 |
| J02984 | SEQ ID NO 105 |
| X87176 | SEQ ID NO 106 |
| M16279 | SEQ ID NO 107 |
| J04208 | SEQ ID NO 108 |
| U79291 | SEQ ID NO 109 |
| Al346190 | SEQ ID NO 110 |
| Al188445 | SEQ ID NO 111 |
| L38961 | SEQ ID NO 112 |
| Al096643 | SEQ ID NO 113 |
| X94453 | SEQ ID NO 114 |
| AB018290 | SEQ ID NO 115 |
| Al681442 | SEQ ID NO 116 |
| X63526 | SEQ ID NO 117 |
| M13450 | SEQ ID NO 118 |
| M61831 | SEQ ID NO 119 |
| M33680 | SEQ ID NO 120 |
| D13639 | SEQ ID NO 121 |
| Al690834 | SEQ ID NO 122 |
| L13278 | SEQ ID NO 123 |
| J03473 | SEQ ID NO 124 |
| D84294 | SEQ ID NO 125 |
| U50939 | SEQ ID NO 126 |
| AF035284 | SEQ ID NO 127 |
| AA843160 | SEQ ID NO 128 |
| L13689 | SEQ ID NO 129 |
| M34480 | SEQ ID NO 130 |
| Al283385 | SEQ ID NO 131 |
| X63657 | SEQ ID NO 132 |
| AA678185 | SEQ ID NO 133 |
| X64229 | SEQ ID NO 134 |
| AF037989 | SEQ ID NO 135 |
| M25753 | SEQ ID NO 136 |
| D38553 | SEQ ID NO 137 |
| Al022085 | SEQ ID NO 138 |
| Al186910 | SEQ ID NO 139 |
| X68060 | SEQ ID NO 140 |
| X70394 | SEQ ID NO 141 |
| Al634838 | SEQ ID NO 142 |
| S78187 | SEQ ID NO 143 |
| Al654133 | SEQ ID NO 144 |
| J02940 | SEQ ID NO 145 |
| Al671161 | SEQ ID NO 146 |
| R55307 | SEQ ID NO 147 |
| AA121546 | SEQ ID NO 148 |
| J03040 | SEQ ID NO 149 |
| AB002352 | SEQ ID NO 150 |
| X65644 | SEQ ID NO 151 |
| U04953 | SEQ ID NO 152 |
| U10323 | SEQ ID NO 153 |
| Al126840 | SEQ ID NO 154 |
| Al697151 | SEQ ID NO 155 |
| U94703 | SEQ ID NO 156 |
| M64571 | SEQ ID NO 157 |
| AB002371 | SEQ ID NO 158 |
| U38847 | SEQ ID NO 159 |
| AB014523 | SEQ ID NO 160 |
| D79988 | SEQ ID NO 161 |
| X82200 | SEQ ID NO 162 |
| X89984 | SEQ ID NO 163 |
| L07555 | SEQ ID NO 164 |
| AF037364 | SEQ ID NO 165 |
| U00947 | SEQ ID NO 166 |
| AA402892 | SEQ ID NO 167 |
| AB011166 | SEQ ID NO 168 |
| Al701109 | SEQ ID NO 169 |
| U41060 | SEQ ID NO 170 |
| AF026293 | SEQ ID NO 171 |
| AF041037 | SEQ ID NO 172 |
| U76421 | SEQ ID NO 173 |
| Z11793 | SEQ ID NO 174 |
| X77794 | SEQ ID NO 175 |
| J00194 | SEQ ID NO 176 |
| J04615 | SEQ ID NO 177 |
| U97105 | SEQ ID NO 178 |
| AF061016 | SEQ ID NO 179 |
| AB006624 | SEQ ID NO 180 |
| U50196 | SEQ ID NO 181 |
| D83777 | SEQ ID NO 182 |
| U75362 | SEQ ID NO 183 |
| D26350 | SEQ ID NO 184 |
| M98343 | SEQ ID NO 185 |
| Al151265 | SEQ ID NO 186 |
| M14745 | SEQ ID NO 187 |
| D50406 | SEQ ID NO 188 |
| Al279820 | SEQ ID NO 189 |
| M57730 | SEQ ID NO 190 |
| U30521 | SEQ ID NO 191 |
| R45293 | SEQ ID NO 192 |
| AF042282 | SEQ ID NO 193 |
| U65410 | SEQ ID NO 194 |
| J04164 | SEQ ID NO 195 |
| AA700158 | SEQ ID NO 196 |
| AF054589 | SEQ ID NO 197 |
| U55206 | SEQ ID NO 198 |
| AF006484 | SEQ ID NO 199 |
| AF062495 | SEQ ID NO 200 |
| U25770 | SEQ ID NO 201 |
| AA829653 | SEQ ID N0 202 |
| D42055 | SEQ ID NO 203 |
| M58459 | SEQ ID NO 204 |
| AA878385 | SEQ ID NO 205 |
| Al191557 | SEQ ID NO 206 |
| AB011004 | SEQ ID NO 207 |
| U92715 | SEQ ID NO 208 |
| L10373 | SEQ ID NO 209 |
| X92814 | SEQ ID NO 210 |
| N39247 | SEQ ID NO 211 |
| AF039022 | SEQ ID NO 212 |
| AB020662 | SEQ ID NO 213 |
| AF009615 | SEQ ID NO 214 |
| AF038953 | SEQ ID NO 215 |
| Al660656 | SEQ ID NO 216 |
| AA192175 | SEQ ID NO 217 |
| M19507 | SEQ ID NO 218 |
| Al142357 | SEQ ID NO 219 |
| AA921856 | SEQ ID NO 220 |
| Al051327 | SEQ ID NO 221 |
| AF006259 | SEQ ID NO 222 |
| D86864 | SEQ ID NO 223 |
| X69804 | SEQ ID NO 224 |
| X82240 | SEQ ID NO 225 |
| X04217 | SEQ ID NO 226 |
| Al357189 | SEQ ID NO 227 |
| S57235 | SEQ ID NO 228 |
| AA926854 | SEQ ID NO 229 |
| L01406 | SEQ ID NO 230 |
| R45298 | SEQ ID NO 231 |
| Y09397 | SEQ ID NO 232 |
| Al336937 | SEQ ID NO 233 |
| U22526 | SEQ ID NO 234 |
| AF088868 | SEQ ID NO 235 |
| AB008913 | SEQ ID NO 236 |
| AB011421 | SEQ ID NO 237 |
| Al005063 | SEQ ID NO 238 |
| J04130 | SEQ ID NO 239 |
| R56094 | SEQ ID NO 240 |
| Al243123 | SEQ ID NO 241 |
| AF091073 | SEQ ID NO 242 |
| U47414 | SEQ ID NO 243 |
| Al650643 | SEQ ID NO 244 |
| Al356773 | SEQ ID NO 245 |
| R39960 | SEQ ID NO 246 |
| AF070587 | SEQ ID NO 247 |
| M17017 | SEQ ID NO 248 |
| AB020663 | SEQ ID NO 249 |
| Al262941 | SEQ ID NO 250 |
| Al262981 | SEQ ID NO 251 |
| AA906175 | SEQ ID NO 252 |
| X75918 | SEQ ID NO 253 |
| AA868968 | SEQ ID NO 254 |
| Al679625 | SEQ ID NO 255 |
| U68019 | SEQ ID NO 256 |
| X04011 | SEQ ID NO 257 |
| X69111 | SEQ ID NO 258 |
| AF097021 | SEQ ID NO 259 |
| AF044288 | SEQ ID NO 260 |
| W84421 | SEQ ID NO 261 |
| U69559 | SEQ ID NO 262 |
| X52195 | SEQ ID NO 263 |
| AF013263 | SEQ ID NO 264 |
| AB014578 | SEQ ID NO 265 |
| Y08136 | SEQ ID NO 266 |
| AF070569 | SEQ ID NO 267 |
| AB018339 | SEQ ID NO 268 |
| U90916 | SEQ ID NO 269 |
| X95239 | SEQ ID NO 270 |
| AF052107 | SEQ ID NO 271 |
| Al656059 | SEQ ID NO 272 |
| A1457525 | SEQ ID NO 273 |
| D86959 | SEQ ID NO 274 |
| D80012 | SEQ ID NO 275 |
| X91249 | SEQ ID NO 276 |
| AF039067 | SEQ ID NO 277 |
| N38966 | SEQ ID NO 278 |
| J05068 | SEQ ID NO 279 |
| AB005047 | SEQ ID NO 280 |
| Z29331 | SEQ ID NO 281 |
| Al479332 | SEQ ID NO 282 |
| Al151509 | SEQ ID NO 283 |
| D86985 | SEQ ID NO 284 |
| L05515 | SEQ ID NO 285 |
| N66072 | SEQ ID NO 286 |
| N57538 | SEQ ID NO 287 |
| Y10313 | SEQ ID NO 288 |
| D10040 | SEQ ID NO 289 |
| AA993127 | SEQ ID NO 290 |
| X89214 | SEQ ID NO 291 |
| AF098642 | SEQ ID NO 292 |
| AF023611 | SEQ ID NO 293 |
| N39237 | SEQ ID NO 294 |
| AB011085 | SEQ ID NO 295 |
| Al223310 | SEQ ID NO 296 |
| AA620747 | SEQ ID NO 297 |
| AF079221 | SEQ ID NO 298 |
| X76061 | SEQ ID NO 299 |
| Al306503 | SEQ ID NO 300 |
| Al268420 | SEQ ID NO 301 |
| Al201868 | SEQ ID NO 302 |
| D87930 | SEQ ID NO 303 |
| AF017995 | SEQ ID NO 304 |
| Y00285 | SEQ ID NO 305 |
| AB014511 | SEQ ID NO 3O6 |
| AF052169 | SEQ ID NO 307 |
| Al344106 | SEQ ID NO 308 |
| Al693930 | SEQ ID NO 309 |
| AA972712 | SEQ ID NO 310 |
| M64673 | SEQ ID NO 311 |
| X90846 | SEQ ID NO 312 |
| L33930 | SEQ ID NO 313 |
| Al052820 | SEQ ID NO 314 |
| Al439194 | SEQ ID NO 315 |
| U31525 | SEQ ID NO 316 |
| AF045459 | SEQ ID NO 317 |
| AA176867 | SEQ ID NO 318 |
| M95767 | SEQ ID NO 319 |
| X58794 | SEQ ID NO 320 |
| Al352299 | SEQ ID NO 321 |
| X54150 | SEQ ID NO 322 |
| AB014536 | SEQ ID NO 323 |
| A1470098 | SEQ ID NO 324 |
| U07139 | SEQ ID NO 325 |
| U08471 | SEQ ID NO 326 |
| AF077346 | SEQ ID NO 327 |
| AB020686 | SEQ ID NO 328 |
| D50840 | SEQ ID NO 329 |
| Al651772 | SEQ ID NO 330 |
| U36336 | SEQ ID NO 331 |
| Al435586 | SEQ ID NO 332 |
| U66672 | SEQ ID NO 333 |
| AF085199 | SEQ ID NO 334 |
| AA485939 | SEQ ID NO 335 |
| AA709067 | SEQ ID NO 336 |
| U67615 | SEQ ID NO 337 |
| X71125 | SEQ ID NO 338 |
| X69910 | SEQ ID NO 339 |
| AF051850 | SEQ ID NO 340 |
| X16354 | SEQ ID NO 341 |
| R59187 | SEQ ID NO 342 |
| J05070 | SEQ ID NO 343 |
| Al354439 | SEQ ID NO 344 |
| D86960 | SEQ ID NO 345 |
| AF034373 | SEQ ID NO 346 |
| AB007918 | SEQ ID NO 347 |
| A1381472 | SEQ ID NO 348 |
| T66135 | SEQ ID NO 349 |
| Al079292 | SEQ ID NO 350 |
| Al091230 | SEQ ID NO 351 |
| Y07759 | SEQ ID NO 352 |
| U79298 | SEQ ID NO 353 |
| AF001434 | SEQ ID NO 354 |
| X89478 | SEQ ID NO 355 |
| AA988547 | SEQ ID NO 356 |
| Al393246 | SEQ ID NO 357 |
| AA961586 | SEQ ID NO 358 |
| H29746 | SEQ ID NO 359 |
| Al493593 | SEQ ID NO 360 |
| D38305 | SEQ ID NO 361 |
| Al378555 | SEQ ID NO 362 |
| Al205344 | SEQ ID NO 363 |
| AA868506 | SEQ ID NO 364 |
| Al673085 | SEQ ID NO 365 |
| U33053 | SEQ ID NO 366 |

In one embodiment, the invention provides a set of 366 gene markers that can classify CML patients as having blast crisis CML (BC-CML) or chronic phase CML (CP-CML). In this respect, the invention provides 366 gene markers able to distinguish whether a patient has progressed from chronic phase to blast crisis. The invention further provides subsets of at least 50, 100, 150, 200, 250 or 300 genetic markers, drawn from the set of 366 markers, which also distinguish blast crisis from chronic phase. The invention also provides a method of using these markers to distinguish between BC-CML and CP-CML patients or cells derived therefrom.

Any of the gene markers provided above may be used alone or with other CML markers, or with markers for other phenotypes or conditions. For example, markers that distinguish CML status may be used in conjunction with those for breast cancer.

### 5.3.2 Identification of Markers

The present invention provides sets of markers for the differentiation of CP-CML samples from BC-CML samples. Generally, the marker sets were identified by determining which of ˜25,000 human markers had expression patters that correlated with the conditions or indications.

In one embodiment, the method for identifying marker sets is as follows. After extraction and labeling of target polynucleotides, the expression of all markers (genes) in a sample is compared to the expression of all markers in a standard or control. The sample may comprise a single sample, or a pool of samples; the samples in the pool may come from different individuals. In one embodiment, the standard or control comprises target polynucleotide molecules derived from a sample from a normal individual (i.e., an individual not afflicted with CML). In a preferred embodiment, the standard or control is a pool of target polynucleotide molecules. The pool may derived from collected samples from a number of normal individuals. In a preferred embodiment, the control pool comprises bone marrow samples taken from a number of individuals having CP-CML. In another preferred embodiment, the pool comprises an artificially-generated population of nucleic acids designed to approximate the level of nucleic acid derived from each marker found in a pool of marker-derived nucleic acids derived from tumor samples.

The comparison may be accomplished by any means known in the art. For example, expression levels of various markers may be assessed by separation of target polynucleotide molecules (e.g., RNA or cDNA) derived from the markers in agarose or polyacrylamide gels, followed by hybridization with marker-specific oligonucleotide probes. Alternatively, the comparison may be accomplished by the labeling of target polynucleotide molecules followed by separation on a sequencing gel. Polynucleotide samples are placed on the gel such that patient and control or standard polynucleotides are in adjacent lanes. Comparison of expression levels is accomplished visually or by means of densitometer. In a preferred embodiment, the expression of all markers is assessed simultaneously by hybridization to an oligonucleotide microarray. In each approach, markers meeting certain criteria are identified as associated with CML.

A marker is selected based upon a significant difference of expression in a sample as compared to a standard or control condition. Selection may be made based upon either significant up- or down regulation of the marker in the patient sample. Selection may also be made by calculation of the statistical significance (i.e., the p-value) of the correlation between the expression of the marker and the condition or indication. Preferably, both selection criteria are used. Thus, in one embodiment of the present invention, markers associated with CML are selected where the markers show both more than two-fold change (increase or decrease) in expression as compared to a standard, and the p-value for the correlation between CML and the change in marker expression is no more than 0.01 (i.e., is statistically significant).

The expression of the identified CML-related markers is then used to identify markers that can differentiate tumors into clinical types. In a specific embodiment using a number of tumor samples, markers are identified by calculation of correlation coefficients between the clinical category and the linear, logarithmic or other transform of expression ratio across all samples for each individual gene. Specifically, the correlation coefficient can be calculated as

ρ=(\vec{c}•\vec{r})/(∥\vec{c}∥·∥\vec{r}∥),

where C represents the category and r represents the linear, logarithmic or any other transform of ratio of expression between sample and control. Markers for which the coefficient of correlation exceeds an arbitrary cutoff are identified as CML-related markers specific for a particular clinical type. In a specific embodiment, markers are chosen if the correlation coefficient is greater than about 0.3 or less than about −0.3.

Next, the significance of the correlation is calculated. This significance may be calculated by any statistical means by which such significance is calculated. In a specific example, a set of correlation data is generated using a Monte-Carlo technique to randomize the association between the expression difference of a particular marker an the clinical category. The frequency distribution of markers satisfying the criteria through calculation of correlation coefficients is compared to the number of markers satisfying the criteria in the data generated through the Monte-Carlo technique. The frequency distribution of markers satisfying the criteria in the Monte-Carlo runs is used to determine whether the number of markers selected by correlation with clinical data is significant. See Example 2.

Once a marker set is identified, the markers may be rank-ordered in order of significance of discrimination. One means of rank ordering is by the amplitude of correlation between the change in gene expression of the marker and the specific condition being discriminated. Another, preferred means is to use a statistical metric. In a specific embodiment, the metric is a Fisher-like statistic:

$$t = \frac{( ⟨ {x}_{1} ⟩ - ⟨ {x}_{2} ⟩ )}{\sqrt{[ {σ}_{1}^{2} ( {n}_{1} - 1 ) + {σ}_{2}^{2} ( {n}_{1} - 1 ) ] / ( {n}_{1} + {n}_{2} - 2 ) / ( 1 / {n}_{1} + 1 / {n}_{2} )}}$$

In this equation, (x₁) is the error-weighted average of the log ratio of transcript expression measurements within the total number of samples, (x₂) is the error-weighted average of log ratio within a first diagnostic group (e.g., BC-CMV), σ₁ is the variance of the log ratio within the total number of samples and n₁ is the number of samples for which valid measurements of log ratios are available. σ₂ is the variance of log ratio within a second, related diagnostic group (e.g., CP-CML), and n₂ is the number of samples for which valid measurements of log ratios are available. The t-value in the above equation represents the variance-compensated difference between two means.

The rank-ordered marker set may be used to optimize the number of markers in the set used for discrimination. This is accomplished generally in a “leave one out” method as follows. In a first run, a subset, for example 5, of the markers is used to generate a template, where out of X samples, X-1 are used to generate the template, and the status of the remaining sample is predicted. In a second run, additional markers, for example 5, area added, so that a template is now generated from 10 markers, and the outcome of the remaining sample is predicted. this process is repeated until the entire set of markers is used to generate the template. For each of the runs, type 1 (false negative) and type 2 (false positive) errors are calculated; the optimal number of markers is that number where the type 1 error rate, type 2 error rate, or, preferably, the total error rate is lowest.

### 5.3.3 Sample Collection

In the present invention, target polynucleotide molecules are extracted from a bone marrow sample taken from an individual afflicted with CML. The sample may be collected in any clinically acceptable manner, but must be collected such that marker-derived polynucleotides (i.e., RNA) are preserved. These polynucleotide molecules are preferably labeled distinguishably from standard or control polynucleotide molecules, and both are hybridized to a microarray comprising some or all of the markers or marker sets or subsets described above. A sample may comprise any clinically relevant tissue sample, such as a bone marrow sample, tumor biopsy, fine needle aspirate, or a sample of bodily fluid, such as blood, plasma, serum, lymph, ascitic fluid, cystic fluid or urine. The sample may be taken from a human, or, in a veterinary context, from non-human animals such as ruminants, horses, swine or sheep, or from domestic companion animals such as felines and canines.

Methods for preparing total and poly(A)+RNA are well known and are described generally in Sambrook et al. (1989, Molecular Cloning—A Laboratory Manual (2nd Ed.), Vols. 1-3, Cold Spring Harbor Laboratory, Cold Spring Harbor, N.Y.) and Ausubel et al., eds. (1994, Current Protocols in Molecular Biology, vol.2, Current Protocols Publishing, New York).

RNA may be isolated from eukaryotic cells by procedures that involve lysis of the cells and denaturation of the proteins contained therein. Cells of interest include wild-type cells (i.e., non-cancerous), drug-exposed wild-type cells, tumor- or tumor-derived cells, modified cells, normal or tumor cell line cells, and drug-exposed modified cells.

Additional steps may be employed to remove DNA. Cell lysis may be accomplished with a nonionic detergent, followed by microcentrifugation to remove the nuclei and hence the bulk of the cellular DNA. In one embodiment, RNA is extracted from cells of the various types of interest using guanidinium thiocyanate lysis followed by CsCl centrifugation to separate the RNA from DNA (Chirgwin et al., 1979, Biochemistry 18:5294-5299). Poly(A)+RNA is selected by selection with oligo-dT cellulose (see Sambrook et al., 1989, Molecular Cloning—A Laboratory Manual (2nd Ed.), Vols. 1-3, Cold Spring Harbor Laboratory, Cold Spring Harbor, N.Y.). Alternatively, separation of RNA from DNA can be accomplished by organic extraction, for example, with hot phenol or phenol/chloroform/isoamyl alcohol.

If desired, RNase inhibitors may be added to the lysis buffer. Likewise, for certain cell types, it may be desirable to add a protein denaturation/digestion step to the protocol.

For many applications, it is desirable to preferentially enrich mRNA with respect to other cellular RNAs, such as transfer RNA (tRNA) and ribosomal RNA (rRNA). Most mRNAs contain a poly(A) tail at their 3′ end. This allows them to be enriched by affinity chromatography, for example, using oligo(dT) or poly(U) coupled to a solid support, such as cellulose or Sephadex™ (see Ausubel et al., eds., 1994, Current Protocols in Molecular Biology, vol. 2, Current Protocols Publishing, New York). Once bound, poly(A)+mRNA is eluted from the affinity column using 2 mM EDTA/0.1% SDS.

The sample of RNA can comprise a plurality of different mRNA molecules, each different mRNA molecule having a different nucleotide sequence. In a specific embodiment, the mRNA molecules in the RNA sample comprise at least 100 different nucleotide sequences.

In a specific embodiment, total RNA or mRNA from cells are used in the methods of the invention. The source of the RNA can be cells of a plant or animal, human, mammal, primate, non-human animal, dog, cat, mouse, rat, bird, yeast, eukaryote, prokaryote, etc. In specific embodiments, the method of the invention is used with a sample containing total mRNA or total RNA from 1×10⁶ cells or less.

### 5.4 Methods of Using CML Marker Sets

### 5.4.1 Diagnostic Methods

The present invention provides for methods of using the marker sets to analyze a sample from an individual so as to determine whether the individual is afflicted with CP-CML or BC-CML. The individual need not, however, actually be afflicted with CML. Essentially, the expression of specific marker genes in the individual, or a sample taken therefrom, is compared to a standard or control. For example, assume two CML-related conditions, X and Y. One can compare the level of expression of CML markers for condition X in an individual to the level of the marker-derived polynucleotides in a control, wherein the level represents the level of expression exhibited by samples having condition X. In this instance, if the expression of the markers in the individual's sample is substantially (i.e., statistically) different from that of the control, then the individual does not have condition X. Where, as here, the choice is bimodal (i.e., a sample is either X or Y), the individual can additionally be said to have condition Y. Of course, the comparison to a control representing condition Y can also be performed. Preferably both are performed simultaneously, such that each control acts as both a positive and a negative control. The distinguishing result may thus either be a demonstrable difference from the expression levels (i.e., the amount of marker-derived RNA, or polynucleotides derived therefrom) represented by the control, or no significant difference.

Thus, in one embodiment, the method of determining a particular tumor-related status of an individual comprises the steps of (1) hybridizing labeled target polynucleotides from an individual to a microarray containing one of the above marker sets; (2) hybridizing standard or control polynucleotides molecules to the microarray, wherein the standard or control molecules are differentially labeled from the target molecules; and (3) determining the difference in transcript levels, or lack thereof, between the target and standard or control, wherein the difference, or lack thereof, determines the individual's CML-related status. In a more specific embodiment, the standard or control molecules comprise marker-derived polynucleotides from a pool of samples from normal individuals, or, preferably, a pool of samples from individuals having blast crisis CML. In another preferred embodiment, the standard or control is an artificially-generated pool of marker-derived polynucleotides, which pool is designed to mimic the level of marker expression exhibited by clinical samples of normal or CML tumor tissue having a particular clinical indication (i. e., CP-CML or BC-CML). In another specific embodiment, the control molecules comprise a pool derived from CML-derived cancer cell lines.

The present invention provides sets of markers useful for distinguishing CP-CML from BC-CML samples. Thus, in one embodiment of the above method, the level of polynucleotides (i.e., mRNA or polynucleotides derived therefrom) in a sample from an individual, expressed from the markers provided in Table 1, are compared to the level of expression of the same markers from a control, wherein the control comprises marker-related polynucleotides derived from chronic phase samples, blast crisis samples, or both. Preferably, the comparison is to both blast crisis samples and chronic phase samples, and preferably the comparison is to polynucleotide pools from a number of CP-CML and BP-CML samples, respectively. Where the individual's marker expression most closely resembles or correlates with the CP-CML control, and does not resemble or correlate with the BP-CML control, the individual is classified as having CML in the chronic phase.

For the above embodiment of the method, the full set of markers may be used (i.e., the complete set of 366 markers listed in Table 1). In other embodiments, subsets of the markers may be used. for example, the subset of markers used may comprise at least 5, 10, 20, 50, 100, 250, or 300 of the marker genes listed in Table 3.

The similarity between the marker expression profile of an individual and that of a control can be assessed a number of ways. In the simplest case, the profiles can be compared visually in a printout of expression difference data. Alternatively, the similarity can be calculated mathematically.

In one embodiment, the similarity measure between two patients x and y, or between patient x and a classifier y, can be calculated using the following equation:

$$S = 1 - [ {\sum }_{t = 1}^{{N}_{v}} \frac{( {x}_{t} - \overline{x} )}{{σ}_{{x}_{t}}} \frac{( {y}_{t} - \overline{y} )}{{σ}_{{y}_{i}}} / \sqrt{{\sum }_{t = 1}^{{N}_{v}} {( \frac{( {x}_{i} - \overline{x} )}{{σ}_{{x}_{t}}} )}^{2} {\sum }_{t = 1}^{{N}_{v}} {( \frac{( {y}_{i} - \overline{y} )}{{σ}_{{y}_{i}}} )}^{2}} ] .$$

In this equation, x and y are two patients with components of log ratio xᵢ and yᵢ, i=1, . . . , N=4,986. Associated with every value xᵢ is error σₓ_{ᵢ}. The smaller the value σₓ_{ᵢ}, the more reliable the measurement

$${x}_{t} \cdot \overline{x} = {\sum }_{i = 1}^{{N}_{v}} \frac{{x}_{t}}{{σ}_{{x}_{t}}^{2}} / {\sum }_{i = 1}^{{N}_{v}} \frac{1}{{σ}_{{x}_{t}}^{2}}$$

is the error-weighted arithmetic mean.

In a preferred embodiment, templates are developed for sample comparison. The template is defined as the error-weighted log ratio average of the expression difference for the group of marker genes able to differentiate the particular CML-related condition (i.e, progression from chronic phase to blast crisis). For example, templates are defined for CP-CML samples and for BC-CML samples. Next, a classifier parameter is calculated. This parameter may be calculated using either expression level differences between the sample and template, or by calculation of a correlation coefficient. Such a coefficient, Pi, can be calculated using the following equation:

Pᵢ=(\vec{z}ᵢ•\vec{y})/(∥\vec{z}ᵢ∥·∥\vec{y}∥),

where zᵢ is the expression template i, and y is the expression profile of a patient.

Thus, in a more specific embodiment, the above method of determining a particular tumor-related status of an individual comprises the steps of (1) hybridizing labeled target polynucleotides from an individual to a microarray containing one of the above marker sets; (2) hybridizing standard or control polynucleotides molecules to the microarray, wherein the standard or control molecules are differentially labeled from the target molecules; and (3) determining the difference in transcript levels, or lack thereof, between the target and standard or control, wherein the control is a template comprising the error-weighted log ratio average of the markers, wherein said determining is accomplished by means of the statistic of Equation 1 or Equation 4, and wherein the difference, or lack thereof, determines the individual's tumor-related status.

### 5.5 Determination of Marker Gene Expression Levels

### 5.5.1 Methods

The expression levels of the marker genes in a sample maybe determined by any means known in the art. The expression level may be determined by isolating and determining the level (i.e., amount) of nucleic acid transcribed from each marker gene. Alternatively, or additionally, the level of specific proteins translated from mRNA transcribed from a marker gene may be determined.

The level of expression of specific marker genes can be accomplished by determining the amount of mRNA, or polynucleotides derived therefrom, present in a sample. Any method for determining RNA levels can be used. For example, RNA is isolated from a sample and separated on an agarose gel. The separated RNA is then transferred to a solid support, such as a filter. Nucleic acid probes representing one or more markers are then hybridized to the filter by northern hybridization, and the amount of marker-derived RNA is determined. Such determination can be visual, or machine-aided, for example, by use of a densitometer. Another method of determining RNA levels is by use of a dot-blot or a slot-blot. In this method, RNA, or nucleic acid derived therefrom, from a sample is labeled. The RNA or nucleic acid derived therefrom is then hybridized to a filter containing oligonucleotides derived from one or more marker genes, wherein the oligonucleotides are placed upon the filter at discrete, easily-identifiable locations. Hybridization, or lack thereof, of the labeled RNA to the filter-bound oligonucleotides is determined visually or by densitometer. Polynucleotides can be labeled using a radiolabel or a fluorescent (i.e., visible) label.

These examples are not intended to be limiting; other methods of determining RNA abundance are known in the art.

The level of expression of particular marker genes may also be assessed by determining the level of the specific protein expressed from the marker genes. This can be accomplished, for example, by separation of proteins from a sample on a polyacrylamide gel, followed by identification of specific marker-derived proteins using antibodies in a western blot. Alternatively, proteins can be separated by two-dimensional gel electrophoresis systems. Two-dimensional gel electrophoresis is well-known in the art and typically involves isoelectric focusing along a first dimension followed by SDS-PAGE electrophoresis along a second dimension. See, e.g., Hames et al., 1990, Gel Electrophoresis of Proteins: A Practical Approach, IRL Press, New York; Shevchenko et al., 1996, Proc. Nat'l Acad. Sci. USA 93:1440-1445; Sagliocco et al., 1996, Yeast 12:1519-1533; Lander, 1996, Science 274:536-539. The resulting electropherograms can be analyzed by numerous techniques, including mass spectrometric techniques, western blotting and immunoblot analysis using polyclonal and monoclonal antibodies.

Alternatively, marker-derived protein levels can be determined by constructing an antibody microarray in which binding sites comprise immobilized, preferably monoclonal, antibodies specific to a plurality of protein species encoded by the cell genome. Preferably, antibodies are present for a substantial fraction of the marker-derived proteins of interest. Methods for making monoclonal antibodies are well known (see, e.g., Harlow and Lane, 1988, Antibodies: A Laboratory Manual, Cold Spring Harbor, New York, which is incorporated in its entirety for all purposes). In a preferred embodiment, monoclonal antibodies are raised against synthetic peptide fragments designed based on genomic sequence of the cell. With such an antibody array, proteins from the cell are contacted to the array, and their binding is assayed with assays known in the art. Generally, the expression, and the level of expression, of proteins of diagnostic or prognostic interest can be detected through immunohistochemical staining of tissue slices or sections.

Finally, expression of marker genes in a number of tissue specimens may be characterized using a “tissue array” (Kononen et al., Nat Med 4(7):844-7 (1998)). In a tissue array, multiple tissue samples are assessed on the same microarray. the arrays allow in situ detection of RNA and protein levels; consecutive sections allow the analysis of multiple samples simultaneously.

### 5.5.2 Microarrays

In preferred embodiments, the methods described herein utilize the markers placed on an oligonucleotide array so that the expression status of each of the markers above is assessed simultaneously. Thus, the invention provides for oligonucleotide arrays comprising each of the marker sets described above (i.e., markers to distinguish CP-CML from BC-CML).

The microarrays provided by the present invention may comprise probes to markers able to distinguish the status of the clinical conditions noted above. In particular, the invention provides oligonucleotide arrays comprising probes to a subset or subsets of at least 5, 10, 25, 50, 100, 200, 300 gene markers, up to the full set of 366 markers, which distinguish CP-CML and BC-CML patients or samples.

General methods pertaining to the construction of microarrays comprising the marker sets and/or subsets above are described in the following sections.

### 5.5.2.1 Cosntruction of Microarrays

Microarrays are prepared by selecting probes which comprise a polynucleotide sequence, and then immobilizing such probes to a solid support or surface. For example, the probes may comprise DNA sequences, RNA sequences, or copolymer sequences of DNA and RNA. The polynucleotide sequences of the probes may also comprise DNA and/or RNA analogues, or combinations thereof. For example, the polynucleotide sequences of the probes may be full or partial fragments of genomic DNA. The polynucleotide sequences of the probes may also be synthesized nucleotide sequences, such as synthetic oligonucleotide sequences. The probe sequences can be synthesized either enzymatically in vivo, enzymatically in vitro (e.g., by PCR), or non-enzymatically in vitro.

The probe or probes used in the methods of the invention are preferably immobilized to a solid support which may be either porous or non-porous. For example, the probes of the invention may be polynucleotide sequences which are attached to a nitrocellulose or nylon membrane or filter covalently at either the 3′ or the 5′ end of the polynucleotide. Such hybridization probes are well known in the art (see, e.g., Sambrook et al., Eds., 1989, Molecular Cloning: A Laboratory Manual, 2nd ed., Vol. 1-3, Cold Spring Harbor Laboratory, Cold Spring Harbor, N.Y.). Alternatively, the solid support or surface may be a glass or plastic surface. In a particularly preferred embodiment, hybridization levels are measured to microarrays of probes consisting of a solid phase on the surface of which are immobilized a population of polynucleotides, such as a population of DNA or DNA mimics, or, alternatively, a population of RNA or RNA mimics. The solid phase may be a nonporous or, optionally, a porous material such as a gel.

In preferred embodiments, a microarray comprises a support or surface with an ordered array of binding (e.g., hybridization) sites or “probes” each representing one of the markers described herein. Preferably the microarrays are addressable arrays, and more preferably positionally addressable arrays. More specifically, each probe of the array is preferably located at a known, predetermined position on the solid support such that the identity (i.e., the sequence) of each probe can be determined from its position in the array (i.e., on the support or surface). In preferred embodiments, each probe is covalently attached to the solid support at a single site.

Microarrays can be made in a number of ways, of which several are described below. However produced, microarrays share certain characteristics. The arrays are reproducible, allowing multiple copies of a given array to be produced and easily compared with each other. Preferably, microarrays are made from materials that are stable under binding (e.g., nucleic acid hybridization) conditions. The microarrays are preferably small, e.g., between 1 cm² and 25 cm², between 12 cm² and 13 cm², or 3 cm². However, larger arrays are also contemplated and may be preferable, e.g., for use in screening arrays. Preferably, a given binding site or unique set of binding sites in the microarray will specifically bind (e.g., hybridize) to the product of a single gene in a cell (e.g., to a specific mRNA, or to a specific cDNA derived therefrom). However, in general, other related or similar sequences will cross hybridize to a given binding site.

The microarrays of the present invention include one or more test probes, each of which has a polynucleotide sequence that is complementary to a subsequence of RNA or DNA to be detected. Preferably, the position of each probe on the solid surface is known. Indeed, the microarrays are preferably positionally addressable arrays. Specifically, each probe of the array is preferably located at a known, predetermined position on the solid support such that the identity (i.e., the sequence) of each probe can be determined from its position on the array (i.e., on the support or surface).

According to the invention, the microarray is an array (i.e., a matrix) in which each position represents one of the markers described herein. For example, each position can contain a DNA or DNA analogue based on genomic DNA to which a particular RNA or cDNA transcribed from that genetic marker can specifically hybridize. The DNA or DNA analogue can be, e.g., a synthetic oligomer or a gene fragment. In one embodiment, probes representing each of the markers is present on the array. In a preferred embodiment, the array comprises at least 5 of the CML gene markers.

### 5.5.2.2 Preparing Probes For Microarrays

As noted above, the “probe” to which a particular polynucleotide molecule specifically hybridizes according to the invention contains a complementary genomic polynucleotide sequence. The probes of the exon profiling array preferably consist of nucleotide sequences of no more than 1,000 nucleotides. In some embodiments, the probes of the exon profiling array consist of nucleotide sequences of 10 to 1,000 nucleotides. In a preferred embodiment, the nucleotide sequences of the probes are in the range of 10-200 nucleotides in length and are genomic sequences of a species of organism, such that a plurality of different probes is present, with sequences complementary and thus capable of hybridizing to the genome of such a species of organism, sequentially tiled across all or a portion of such genome. In other specific embodiments, the probes are in the range of 10-30 nucleotides in length, in the range of 10-40 nucleotides in length, in the range of 20-50 nucleotides in length, in the range of 40-80 nucleotides in length, in the range of 50-150 nucleotides in length, in the range of 80-120 nucleotides in length, and most preferably are 60 nucleotides in length.

The probes may comprise DNA or DNA “mimics” (e.g., derivatives and analogues) corresponding to a portion of an organism's genome. In another embodiment, the probes of the microarray are complementary RNA or RNA mimics. DNA mimics are polymers composed of subunits capable of specific, Watson-Crick-like hybridization with DNA, or of specific hybridization with RNA. The nucleic acids can be modified at the base moiety, at the sugar moiety, or at the phosphate backbone. Exemplary DNA mimics include, e.g., phosphorothioates.

DNA can be obtained, e.g., by polymerase chain reaction (PCR) amplification of genomic DNA or cloned sequences. PCR primers are preferably chosen based on a known sequence of the genome that will result in amplification of specific fragments of genomic DNA. Computer programs that are well known in the art are useful in the design of primers with the required specificity and optimal amplification properties, such as Oligo version 5.0 (National Biosciences). Typically each probe on the microarray will be between 10 bases and 50,000 bases, usually between 300 bases and 1,000 bases in length. PCR methods are well known in the art, and are described, for example, in Innis et al., eds., 1990, PCR Protocols: A Guide to Methods and Applications, Academic Press Inc., San Diego, Calif. It will be apparent to one skilled in the art that controlled robotic systems are useful for isolating and amplifying nucleic acids.

An alternative, preferred means for generating the polynucleotide probes of the microarray is by synthesis of synthetic polynucleotides or oligonucleotides, e.g., using N-phosphonate or phosphoramidite chemistries (Froehler et al., 1986, Nucleic Acid Res. 14:5399-5407; McBride et al., 1983, Tetrahedron Lett. 24:246-248). Synthetic sequences are typically between about 10 and about 500 bases in length, more typically between about 20 and about 100 bases, and most preferably between about 40 and about 70 bases in length. In some embodiments, synthetic nucleic acids include non-natural bases, such as, but by no means limited to, inosine. As noted above, nucleic acid analogues may be used as binding sites for hybridization. An example of a suitable nucleic acid analogue is peptide nucleic acid (see, e.g., Egholm et al., 1993, Nature 363:566-568; U.S. Pat. No. 5,539,083).

Probes are preferably selected using an algorithm that takes into account binding energies, base composition, sequence complexity, cross-hybridization binding energies, and secondary structure (see Friend et al., International Patent Publication WO 01/05935, published Jan. 25, 2001).

A skilled artisan will also appreciate that positive control probes, e.g., probes known to be complementary and hybridizable to sequences in the target polynucleotide molecules, and negative control probes, e.g., probes known to not be complementary and hybridizable to sequences in the target polynucleotide molecules, should be included on the array. In one embodiment, positive controls are synthesized along the perimeter of the array. In another embodiment, positive controls are synthesized in diagonal stripes across the array. In still another embodiment, the reverse complement for each probe is synthesized next to the position of the probe to serve as a negative control. In yet another embodiment, sequences from other species of organism are used as negative controls or as “spike-in” controls.

### 5.5.2.3 Attaching Probes to the Solid Surface

The probes are attached to a solid support or surface, which may be made, e.g., from glass, plastic (e.g., polypropylene, nylon), polyacrylamide, nitrocellulose, gel, or other porous or nonporous material. A preferred method for attaching the nucleic acids to a surface is by printing on glass plates, as is described generally by Schena et al., 1995, Science 270:467-470. This method is especially useful for preparing microarrays of cDNA (See also, DeRisi et al., 1996, Nature Genetics 14:457-460; Shalon et al., 1996, Genome Res. 6:639-645; and Schena et al., 1995, Proc. Natl. Acad. Sci. U.S.A. 93:10539-11286).

A second preferred method for making microarrays is by making high-density oligonucleotide arrays. Techniques are known for producing arrays containing thousands of oligonucleotides complementary to defined sequences, at defined locations on a surface using photolithographic techniques for synthesis in situ (see, Fodor et al., 1991, Science 251:767-773; Pease et al., 1994, Proc. Natl. Acad. Sci. U.S.A. 91:5022-5026; Lockhart et al., 1996, Nature Biotechnology 14:1675; U.S. Pat. Nos. 5,578,832; 5,556,752; and 5,510,270) or other methods for rapid synthesis and deposition of defined oligonucleotides (Blanchard et al., Biosensors & Bioelectronics 11:687-690). When these methods are used, oligonucleotides (e.g., 60-mers) of known sequence are synthesized directly on a surface such as a derivatized glass slide. Usually, the array produced is redundant, with several oligonucleotide molecules per RNA.

Other methods for making microarrays, e.g., by masking (Maskos and Southern, 1992, Nuc. Acids. Res. 20:1679-1684), may also be used. In principle, and as noted supra, any type of array, for example, dot blots on a nylon hybridization membrane (see Sambrook et al., supra) could be used. However, as will be recognized by those skilled in the art, very small arrays will frequently be preferred because hybridization volumes will be smaller.

In one embodiment, the arrays of the present invention are prepared by synthesizing polynucleotide probes on a support. In such an embodiment, polynucleotide probes are attached to the support covalently at either the 3′ or the 5′ end of the polynucleotide.

In a particularly preferred embodiment, microarrays of the invention are manufactured by means of an ink jet printing device for oligonucleotide synthesis, e.g., using the methods and systems described by Blanchard in U.S. Pat. No.6,028,189; Blanchard et al., 1996, Biosensors and Bioelectronics 11:687-690; Blanchard, 1998, in Synthetic DNA Arrays in Genetic Engineering, Vol.20, J. K. Setlow, Ed., Plenum Press, New York at pages 111-123. Specifically, the oligonucleotide probes in such microarrays are preferably synthesized in arrays, e.g., on a glass slide, by serially depositing individual nucleotide bases in “microdroplets” of a high surface tension solvent such as propylene carbonate. The microdroplets have small volumes (e.g., 100 pL or less, more preferably 50 pL or less) and are separated from each other on the microarray (e.g., by hydrophobic domains) to form circular surface tension wells which define the locations of the array elements (i.e., the different probes). Microarrays manufactured by this ink-jet method are typically of high density, preferably having a density of at least about 2,500 different probes per 1 cm². The polynucleotide probes are attached to the support covalently at either the 3′ or the 5′ end of the polynucleotide.

### 5.5.2.4 Target Polynucleotide Molecules

The polynucleotide molecules which may be analyzed by the present invention (the “target polynucleotide molecules”) may be from any clinically relevant source, but are expressed RNA or a nucleic acid derived therefrom (e.g., cDNA or amplified RNA derived from cDNA that incorporates an RNA polymerase promoter), including naturally occurring nucleic acid molecules, as well as synthetic nucleic acid molecules. In one embodiment, the target polynucleotide molecules comprise RNA, including, but by no means limited to, total cellular RNA, poly(A)⁺ messenger RNA (mRNA) or fraction thereof, cytoplasmic mRNA, or RNA transcribed from cDNA (i.e., cRNA; see, e.g., Linsley & Schelter, U.S. patent application Ser. No. 09/411,074, filed Oct. 4, 1999, or U.S. Pat. Nos. 5,545,522, 5,891,636, or 5,716,785). Methods for preparing total and poly(A)⁺ RNA are well known in the art, and are described generally, e.g., in Sambrook et al., supra. In one embodiment, RNA is extracted from cells of the various types of interest in this invention using guanidinium thiocyanate lysis followed by CsCl centrifugation (Chirgwin et al., 1979, Biochemistry 18:5294-5299). In another embodiment, total RNA is extracted using a silica gel-based column, commercially available examples of which include RNeasy (Qiagen, Valencia, Calif.) and StrataPrep (Stratagene, La Jolla, Calif.). In an alternative embodiment, which is preferred for S. cerevisiae, RNA is extracted from cells using phenol and chloroform, as described in Ausubel et al., (Ausubel et al., eds., 1989, Current Protocols in Molecular Biology, Vol III, Green Publishing Associates, Inc., John Wiley & Sons, Inc., New York, at pp. 13.12.1-13.12.5). Poly(A)⁺ RNA can be selected, e.g., by selection with oligo-dT cellulose or, alternatively, by oligo-dT primed reverse transcription of total cellular RNA. In one embodiment, RNA can be fragmented by methods known in the art, e.g., by incubation with ZnCl₂, to generate fragments of RNA. In another embodiment, the polynucleotide molecules analyzed by the invention comprise cDNA, or PCR products of amplified RNA or cDNA.

In one embodiment, total RNA, mRNA, or nucleic acids derived therefrom, from a sample taken from a person afflicted with CML. Target polynucleotide molecules that are poorly expressed in particular cells may be enriched using normalization techniques (Bonaldo et al., 1996, Genome Res. 6:791-806).

As described above, the target polynucleotides are detectably labeled at one or more nucleotides. Any method known in the art may be used to detectably label the target polynucleotides. Preferably, this labeling incorporates the label uniformly along the length of the RNA, and more preferably, the labeling is carried out at a high degree of efficiency. One embodiment for this labeling uses oligo-dT primed reverse transcription to incorporate the label; however, conventional methods of this method are biased toward generating 3′ end fragments. Thus, in a preferred embodiment, random primers (e.g., 9-mers) are used in reverse transcription to uniformly incorporate labeled nucleotides over the full length of the target polynucleotides. Alternatively, random primers may be used in conjunction with PCR methods or T7 promoter-based in vitro transcription methods in order to amplify the target polynucleotides.

In a preferred embodiment, the detectable label is a luminescent label. For example, fluorescent labels, bio-luminescent labels, chemi-luminescent labels, and colorimetric labels may be used in the present invention. In a highly preferred embodiment, the label is a fluorescent label, such as a fluorescein, a phosphor, a rhodamine, or a polymethine dye derivative. Examples of commercially available fluorescent labels include, for example, fluorescent phosphoramidites such as FluorePrime (Amersham Pharmacia, Piscataway, N.J.), Fluoredite (Millipore, Bedford, Mass.), FAM (ABI, Foster City, Calif.), and Cy3 or Cy5 (Amersham Pharmacia, Piscataway, N.J.). In another embodiment, the detectable label is a radiolabeled nucleotide.

In a further preferred embodiment, target polynucleotide molecules from a patient sample are labeled differentially from target polynucleotide molecules of a standard. The standard can comprise target polynucleotide molecules from normal individuals (i.e., those not afflicted with CML). In a highly preferred embodiment, the standard comprises target polynucleotide molecules pooled from samples from normal individuals or cell samples from individuals exhibiting chronic phase CML. In another embodiment, the target polynucleotide molecules are derived from the same individual, but are taken at different time points, and thus indicate the efficacy of a treatment by a change in expression of the markers, or lack thereof, during and after the course of treatment (i.e., chemotherapy, radiation therapy or cryotherapy), wherein a change in the expression of the markers from a blast crisis pattern to a chronic phase pattern indicates that the treatment is efficacious. In this embodiment, different timepoints are differentially labeled.

### 5.5.2.5 Hybridization to Microarrays

Nucleic acid hybridization and wash conditions are chosen so that the target polynucleotide molecules specifically bind or specifically hybridize to the complementary polynucleotide sequences of the array, preferably to a specific array site, wherein its complementary DNA is located.

Arrays containing double-stranded probe DNA situated thereon are preferably subjected to denaturing conditions to render the DNA single-stranded prior to contacting with the target polynucleotide molecules. Arrays containing single-stranded probe DNA (e.g., synthetic oligodeoxyribonucleic acids) may need to be denatured prior to contacting with the target polynucleotide molecules, e.g., to remove hairpins or dimers which form due to self complementary sequences.

Optimal hybridization conditions will depend on the length (e.g., oligomer versus polynucleotide greater than 200 bases) and type (e.g., RNA, or DNA) of probe and target nucleic acids. One of skill in the art will appreciate that as the oligonucleotides become shorter, it may become necessary to adjust their length to achieve a relatively uniform melting temperature for satisfactory hybridization results. General parameters for specific (i.e., stringent) hybridization conditions for nucleic acids are described in Sambrook et al., (supra), and in Ausubel et al., 1987, Current Protocols in Molecular Biology, Greene Publishing and Wiley-Interscience, New York. Typical hybridization conditions for the cDNA microarrays of Schena et al., are hybridization in 5×SSC plus 0.2% SDS at 65° C. for four hours, followed by washes at 25° C. in low stringency wash buffer (1×SSC plus 0.2% SDS), followed by 10 minutes at 25° C. in higher stringency wash buffer (0.1×SSC plus 0.2% SDS) (Shena et al., 1996, Proc. Natl. Acad. Sci. U.S.A. 93:10614). Useful hybridization conditions are also provided in, e.g., Tijessen, 1993, Hybridization With Nucleic Acid Probes, Elsevier Science Publishers B.V.; and Kricka, 1992, Nonisotopic DNA Probe Techniques, Academic Press, San Diego, Calif.

Particularly preferred hybridization conditions include hybridization at a temperature at or near the mean melting temperature of the probes (e.g., within 5° C., more preferably within 2° C.) in 1 M NaCl, 50 mM MES buffer (pH 6.5),0.5% sodium sarcosine and 30% formamide.

### 5.5.2.6 Signal Detection and Data Analysis

When fluorescently labeled probes are used, the fluorescence emissions at each site of a microarray may be, preferably, detected by scanning confocal laser microscopy. In one embodiment, a separate scan, using the appropriate excitation line, is carried out for each of the two fluorophores used. Alternatively, a laser may be used that allows simultaneous specimen illumination at wavelengths specific to the two fluorophores and emissions from the two fluorophores can be analyzed simultaneously (see Shalon et al., 1996, A DNA microarray system for analyzing complex DNA samples using two-color fluorescent probe hybridization, Genome Research 6:639-645, which is incorporated by reference in its entirety for all purposes). In a preferred embodiment, the arrays are scanned with a laser fluorescent scanner with a computer controlled X-Y stage and a microscope objective. Sequential excitation of the two fluorophores is achieved with a multi-line, mixed gas laser and the emitted light is split by wavelength and detected with two photomultiplier tubes. Fluorescence laser scanning devices are described in Schena et al., 1996, Genome Res. 6:639-645 and in other references cited herein. Alternatively, the fiber-optic bundle described by Ferguson et al., 1996, Nature Biotech. 14:1681-1684, may be used to monitor mRNA abundance levels at a large number of sites simultaneously.

Signals are recorded and, in a preferred embodiment, analyzed by computer, e.g., using a 12 bit analog to digital board. In one embodiment the scanned image is despeckled using a graphics program (e.g., Hijaak Graphics Suite) and then analyzed using an image gridding program that creates a spreadsheet of the average hybridization at each wavelength at each site. If necessary, an experimentally determined correction for “cross talk” (or overlap) between the channels for the two fluors may be made. For any particular hybridization site on the transcript array, a ratio of the emission of the two fluorophores can be calculated. The ratio is independent of the absolute expression level of the cognate gene, but is useful for genes whose expression is significantly modulated in association with the different CML-related condition.

### 5.6 Computer-Facilitated Analysis

The present invention further provides for kits comprising the marker sets above. In a preferred embodiment, the kit contains a microarray ready for hybridization to target polynucleotide molecules, plus software for the data analyses described above.

The analytic methods described in the previous sections can be implemented by use of the following computer systems and according to the following programs and methods. A Computer system comprises internal components linked to external components. The internal components of a typical computer system include a processor element interconnected with a main memory. For example, the computer system can be an Intel 8086-,80386-,80486-, Pentium™, or Pentium™-based processor with preferably 32 MB or more of main memory.

The external components may include mass storage. This mass storage can be one or more hard disks (which are typically packaged together with the processor and memory). Such hard disks are preferably of 1 GB or greater storage capacity. Other external components include a user interface device, which can be a monitor, together with an inputting device, which can be a mouse, or other graphic input devices, and/or a keyboard. A printing device can also be attached to the computer.

Typically, a computer system is also linked to network link, which can be part of an Ethernet link to other local computer systems, remote computer systems, or wide area communication networks, such as the Internet. This network link allows the computer system to share data and processing tasks with other computer systems.

Loaded into memory during operation of this system are several software components, which are both standard in the art and special to the instant invention. These software components collectively cause the computer system to function according to the methods of this invention. These software components are typically stored on the mass storage device. A software component comprises the operating system, which is responsible for managing computer system and its network interconnections. This operating system can be, for example, of the Microsoft Windows® family, such as Windows 3.1, Windows 95, Windows 98, Windows 2000 or Windows NT. The software component represents common languages and functions conveniently present on this system to assist programs implementing the methods specific to this invention. Many high or low level computer languages can be used to program the analytic methods of this invention. Instructions can be interpreted during run-time or compiled. Preferred languages include C/C++, FORTRAN and JAVA. Most preferably, the methods of this invention are programmed in mathematical software packages that allow symbolic entry of equations and high-level specification of processing, including algorithms to be used, thereby freeing a user of the need to procedurally program individual equations or algorithms. Such packages include Matlab from Mathworks (Natick, Mass.), Mathematica® from Wolfram Research (Champaign, Ill.), or S-Plus® from Math Soft (Cambridge, Mass.). Specifically, the software component includes the analytic methods of the invention as programmed in a procedural language or symbolic package.

The software to be included with the kit comprises the data analysis methods of the invention as disclosed herein. In particular, the software may include mathematical routines for marker discovery, including the calculation of correlation coefficients between clinical categories (i.e., ER status) and marker expression. The software may also include mathematical routines for calculating the correlation between sample marker expression and control marker expression, using array-generated fluorescence data, to determine the clinical classification of a sample.

In an exemplary implementation, to practice the methods of the present invention, a user first loads experimental data into the computer system. These data can be directly entered by the user from a monitor, keyboard, or from other computer systems linked by a network connection, or on removable storage media such as a CD-ROM, floppy disk (not illustrated), tape drive (not illustrated), ZIP® drive (not illustrated) or through the network. Next the user causes execution of expression profile analysis software which performs the methods of the present invention.

In another exemplary implementation, a user first loads experimental data and/or databases into the computer system. This data is loaded into the memory from the storage media or from a remote computer, preferably from a dynamic geneset database system, through the network. Next the user causes execution of software that performs the steps of the present invention.

Alternative computer systems and software for implementing the analytic methods of this invention will be apparent to one of skill in the art and are intended to be comprehended within the accompanying claims. In particular, the accompanying claims are intended to include the alternative program structures for implementing the methods of this invention that will be readily apparent to one of skill in the art.

### 1. EXAMPLES

Materials and Methods

Two analytical methods were used in the present study. The first one involves the examination of the gene expression patterns from all samples by unsupervised clustering to identify the dominant classes. The second one concentrates on the identification of a set of marker genes for the CML progression and the progression classification of samples based on the set of marker genes.

1. Sample Collection

Nineteen cases of chronic phase (n=12) and blast crisis (n=7) CML were randomly selected from archival samples obtained from patients seen at the Fred Hutchinson Cancer Research Center. Status of disease was based on morphology, flow cytometry, cytogenetics, and clinical history. The ages of the patients selected ranged from 30-50 years of age.

2. Amplification, Labeling, and Hybridization

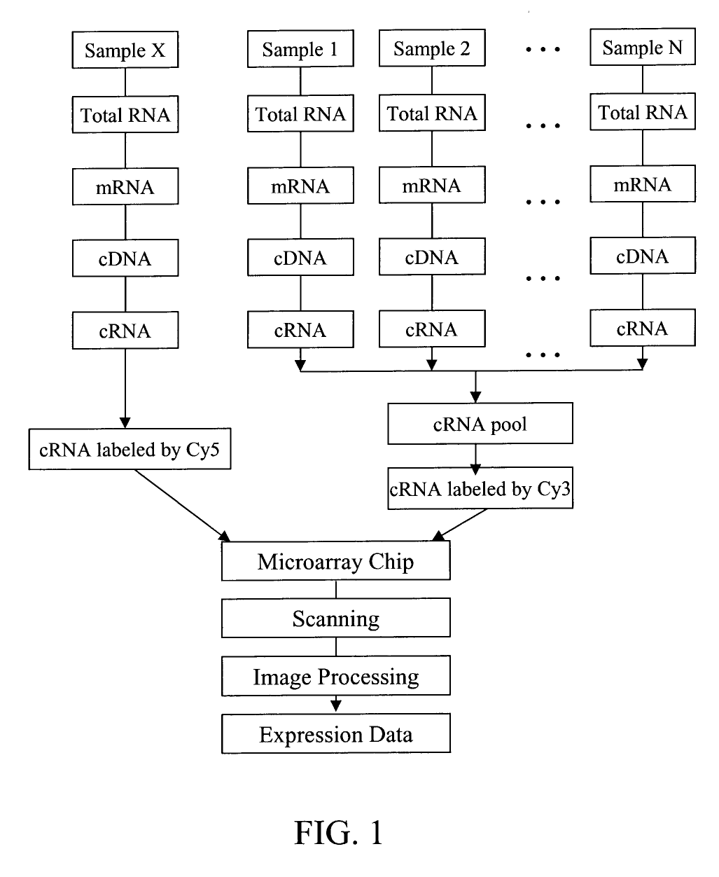

As shown in FIG. 1, total RNA was extracted from fresh bone marrow cells of CML patients by using RNeasy columns (Qiagen). 3′-end cDNA was synthesized by an adaptation of the protocol of Zhao et al., (see, Biotechniques 24:842-852 (1998)). To prevent transcript detection biases stemming from unequal amplification of certain sequences during PCR, the amount of input RNA was increased to 3mg and the number of PCR cycles was decreased to 10. To allow further sequence amplification by cRNA synthesis, a T7RNAP promoter sequence was added to the 3′-end primer sequence used during PCR. Following PCR, amplified DNA was isolated by phenol/chloroform extraction and then transcribed into cRNA by T7RNAP in an in vitro transcription (IVT) reaction (MegaScript, Ambion). cRNA was labeled with Cy3 or Cy5 dyes using a two-step process. First, allylamine-derivitized nucleotides were enzymatically incorporated into cRNA products. For cRNA labeling, a 3:1 mixture of 5-(3-Aminoallyl)uridine 5′-triphosphate (Sigma) and UTP was substituted for UTP in the IVT reaction. Allylamine-derivitized cRNA products were then reacted with N-hydroxy succinimide esters of Cy3 or Cy5 (CyDye, Amersham Pharmacia Biotech). 5 μg Cy5-labeled cRNA from CML patient were mixed with the same amount of Cy3-labeled product from the pool of equal amount of cRNA from each chronic phase CML patient. Hybridizations were done in duplicate with fluor reversals. Before hybridization, labeled cRNAs were fragmented to an average size of ˜50-100 nt by heating at 60° C. in the presence of 10 mM ZnCl₂. Fragmented cRNAs were added to hybridization buffer containing 1 M NaCl, 0.5% sodium sarcosine and 50 mM MES, pH 6.5, which stringency was regulated by the addition of formamide to a final concentration of 30%. Hybridizations were carried out in a final volume of 3 mls at 40° C. on a rotating platform in a hybridization oven (Robbins Scientific). After hybridization, slides were washed and scanned using a confocal laser scanner (Agilent Technologies). Fluorescence intensities on scanned images were quantified, normalized and corrected (see, Hughes at al., 2001, Nature Biotechnology 19:342-347)

3. Pooling of Samples

The reference cRNA pool was formed by pooling equal amount of cRNAs from each chronic phase CML patient. There were cRNAs from 12 patients in this pool.

4. 25 k Human Microarray

Surface-bound oligo nucleotides were synthesized essentially as proposed by Blanchard et al., (see, e.g., Blanchard, International Patent Publication WO 89/41531, published Sep. 24, 1998; Blanchard et al., 1996, Biosensors and Bioelectronics 11:687-690; Blanchard, 1998, in Synthetic DNA Arrays in Genetic Engineering, Vol. 20, J. K. Setlow, Ed., Plenum Press, New York at pages 111-123). Hydrophobic glass surfaces (3 inches by 3 inches) containing exposed hydroxyl groups and used as substrates for nucleotide synthesis. Phosphoramidite monomers were delivered to computer-defined positions on the glass surfaces using ink-jet printer heads. Unreacted monomers were then washed away and the ends of the extended oligonucleotides were deprotected. This cycle of monomer coupling, washing and deprotection was repeated for each desired layer of nucleotide synthesis. Oligonucleotide sequences to be printed were specified by computer files.

Hu25K microarrays represented the ˜25,000 oligonucleotides were used for this study. Sequences for microarrays were selected from the longest messenger RNA (mRNA) sequences representing UniGene clusters (Release 111, Apr. 15, 1999) (available on the Internet at ncbi.nlm.nih.gov/UniGene/). Each mRNA or EST contig was represented on Hu25K microarray by a single 60 mer oligonucleotide chosen by oligo probe design program.

### Example 1

### Identification of Markers Associated with Chronic Myeloid Leukemia

Of ˜25,000 sequences represented on the microarray, a group of 245 genes that were significantly regulated between the BC patients and the CP patients were selected based on the BC pool vs CP pool profile. A gene is determined to be a significant gene if it was differentially regulated with the p-value of differential regulation significance less than 0.001 either upwards or downwards in this BC pool vs CP pool experiment.

An unsupervised clustering algorithm allowed us to cluster patients based on their similarities measured over this set of 245 significant genes. The similarity measure between two patients x and y is defined as

$$S = 1 - [ {\sum }_{i = 1}^{{N}_{v}} \frac{( {x}_{i} - \overline{x} )}{{σ}_{{x}_{t}}} \frac{( {y}_{i} - \overline{y} )}{{σ}_{{y}_{t}}} / \sqrt{{\sum }_{i = 1}^{{N}_{v}} {( \frac{( {x}_{i} - \overline{x} )}{{σ}_{{x}_{i}}} )}^{2} {\sum }_{i = 1}^{{N}_{v}} {( \frac{( {y}_{i} - \overline{y} )}{{σ}_{{y}_{t}}} )}^{2}} ] \qquad ( 1 )$$

In Equation (1), x and y are two patients with components of log ratio xᵢ and yᵢ, i=1, . . . , N=4,986. Associated with every value xᵢ is error σₓ_{ᵢ}. The smaller the value σₓ_{ᵢ}, the more reliable the measurement.

$${x}_{t} \cdot \overline{x} = {\sum }_{i = 1}^{{N}_{v}} \frac{{x}_{t}}{{σ}_{{x}_{t}}^{2}} / {\sum }_{i = 1}^{{N}_{v}} \frac{1}{{σ}_{{x}_{t}}^{2}}$$

is the error-weighted arithmetic mean. The use of correlation as similarity metric emphasizes the importance of co-regulation in clustering rather than the amplitude of regulations.

The set of 245 genes can also be clustered based on their similarities measured over the group of 20 experiments. The similarity measure between two genes is defined in the same way as in Equation (1) except that now for each gene, there are 20 components of log ratio measurements.

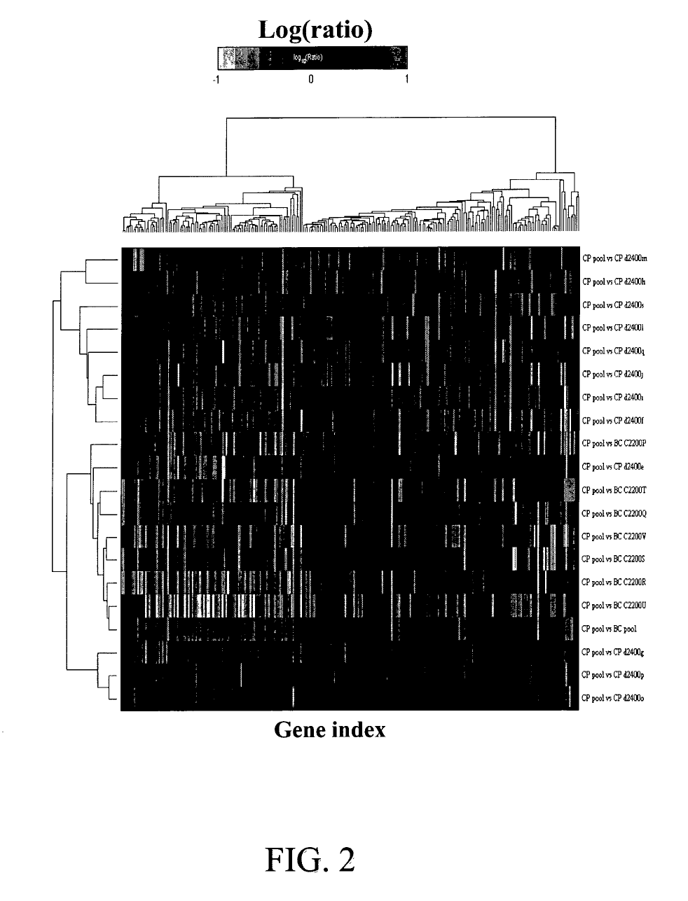

The result of such a two-dimensional clustering is displayed in FIG. 2. Two distinctive patterns are remarkably noticeable in FIG. 2. The first one consists of a group of 8 experiments in the lower part of the plot whose regulations are not very different from the pool made of patients in chronic phase. The other pattern consists of a group of 12 experiments in the upper part of the plot whose expression are substantially different from the pool made of patients in chronic phase. These dominant patterns suggest that the samples can be unambiguously divided into two distinct types based on this set of 245 significant genes. Indeed, 8 samples in the first group are found to be from chronic phase patients. It was also found that 6 samples in the second group are those from blast crisis patients and 6 samples are those clinically known as chronic phase. Our analysis has revealed one case that was classified as morphologically defined chronic phase, more closely resembles blast crisis rather than chronic phase. This patient tended to have other laboratory data suggestive of progression.

From FIG. 2, it was concluded that gene expression patterns can be used to classify CML samples into subgroups of progression as we expected. Supervised statistical methods were then used to identify a set of marker genes which in turn could be used to assess the CML progression.

### Example 2

### Identification of Genetic Markers Expressed in the Progression From Chronic Phase to Blast Crisis in CML

1. Selection of Candidate Discriminating Genes

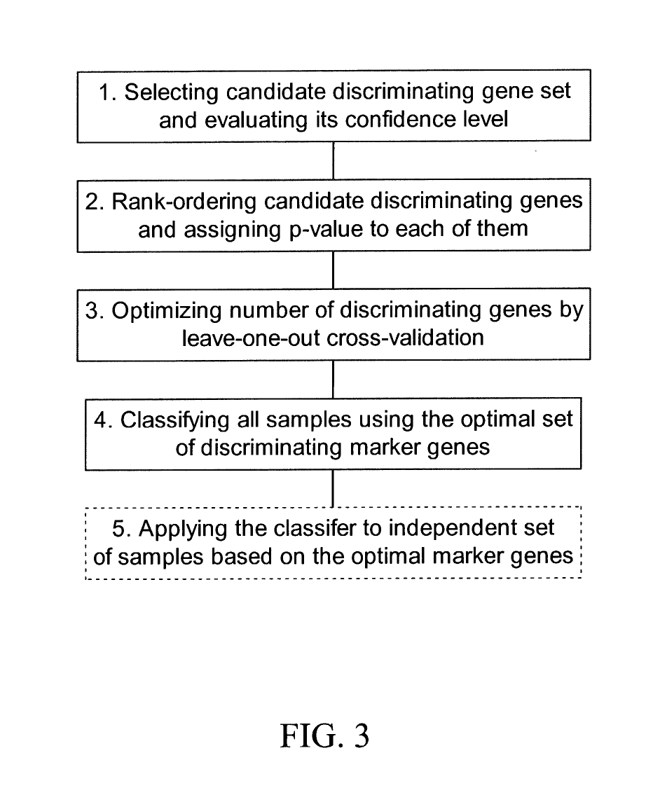

The procedure for marker discovery is outlined in FIG. 3. In the first step, a set of candidate discriminating genes was identified based on gene expression data of training samples. Six patients in the BC group and 8 patients in the CP group were used for training. Specifically, a metric similar to “Fisher” statistic was calculated:

$$t = \frac{( ⟨ {x}_{1} ⟩ - ⟨ {x}_{2} ⟩ )}{\sqrt{[ {σ}_{1}^{2} ( {n}_{1} - 1 ) + {σ}_{2}^{2} ( {n}_{1} - 1 ) ] / ( {n}_{1} + {n}_{2} - 1 ) / ( 1 / {n}_{1} + 1 / {n}_{2} )}} \qquad ( 2 )$$

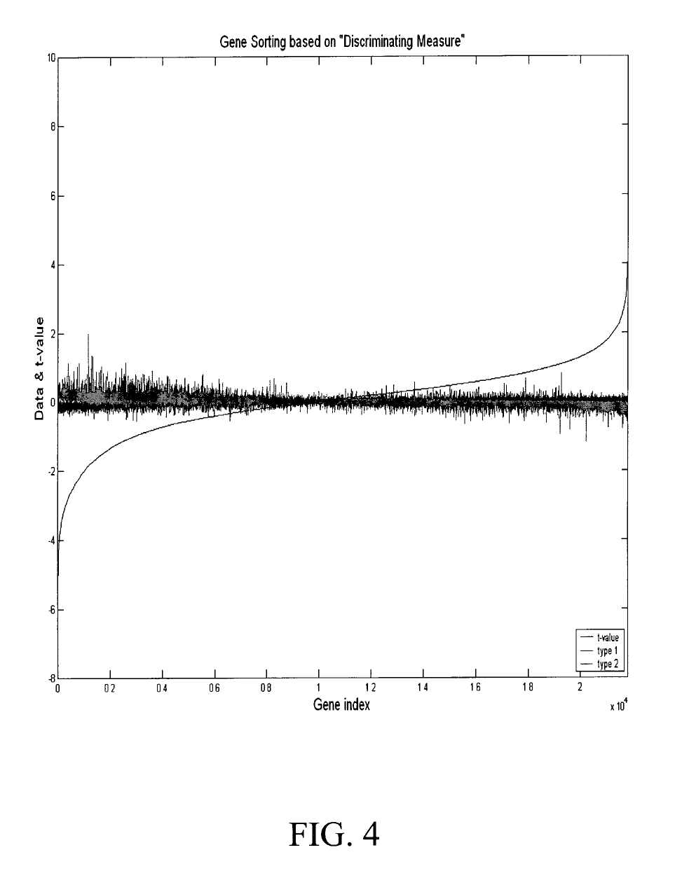

In Equation (2), (x₁) is the error-weighted average of log ratio within the “CP” group and (x₂) is the error-weighted average of log ratio within the “BC” group. σ₁ is the variance of log ratio within the “CP” group and n₁ is the number of samples that we had valid measurements of log ratios. σ₂ is the variance of log ratio within the “BC” group and n₂ is the number of samples that we had valid measurements of log ratios. t-value in Equation (2) presents the variance-compensated difference between two means. Results of t-value for each gene are shown in FIG. 4, together with (x₁) and (x₂).

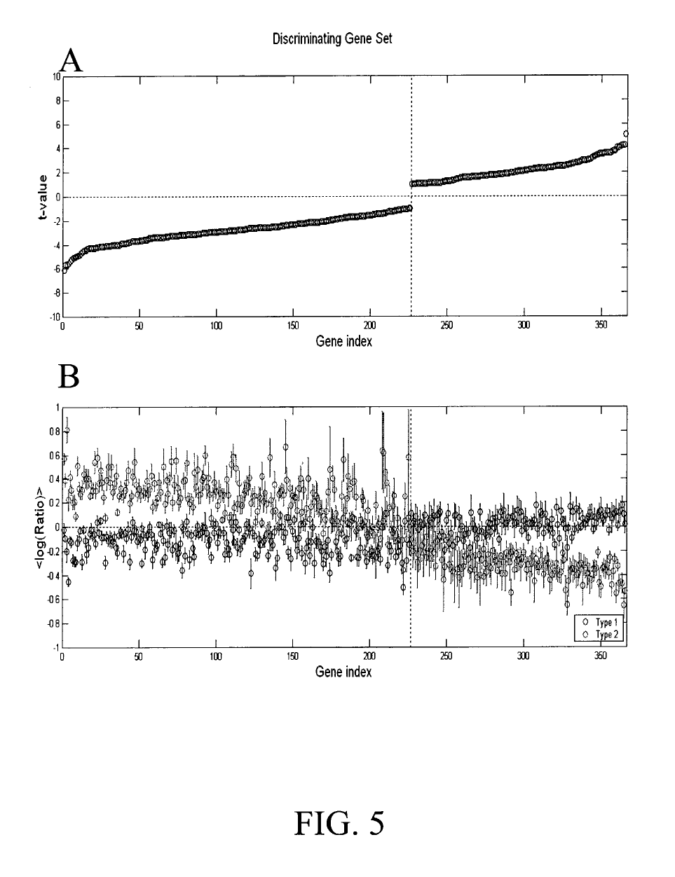

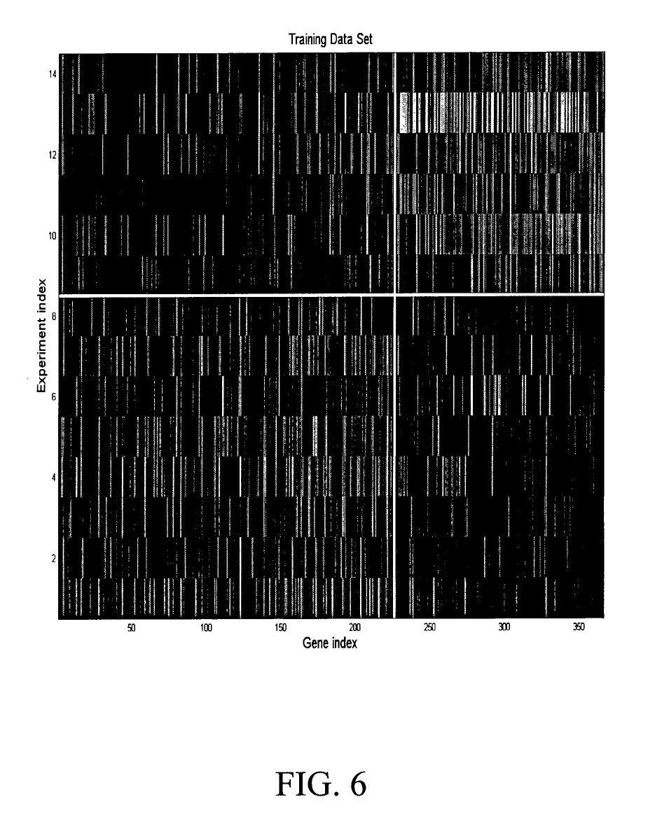

A group of 366 discriminating genes were finally selected by applying a series of cuts to the data including log(Ratio)|>0.3, p<0.01 in at least 2 experiments and |t|>1. The confidence level of each gene in the this list was estimated with respect to a null hypothesis derived from the actual data set using the bootstrap technique. The t-value, averaged log ratio in BC group, averaged log ratio in PC group are shown for these selected genes in FIGS. 5A and 5B. From FIG. 5A, it is clear that on average the expressions of the two groups are dramatically different for the selected genes. FIG. 6 shows the behaviors of each individual sample over this set of marker genes. Table 1 lists all of these 366 marker genes, together with the available information such as their gene descriptions and their functions.

Many of marker genes that were identified have not been known previously to have associations with CML. These genes include numerous numbers of ESTs. This group of genes was ranked by confidence level or t-value in Equation (2).

2. Classification of CML Patients Based on Marker Genes

In the second step, a set of classifier parameters was calculated for each type of training data sets based on either correlation or distance. In particular, a template for the CP group (called \vec{z}₁) was defined by using the error-weighted log ratio average of the selected group of genes. Similarly, we defined a template for the BC group (called \vec{z}₂) by using the error-weighted log ratio average of the selected group of genes. Two classifier parameters (P₁ and P₂) were defined based on either correlation or distance. P₁ measures the similarity between one sample \vec{y} and the “CP” template \vec{z} ₁ over this selected group of genes. P₂ measures the similarity between one sample \vec{y} and the BC template \vec{z}₂ over this selected group of genes. The correlation Pi is defined as:

Pᵢ=(\vec{z}ᵢ•\vec{y})/(∥\vec{z}ᵢ∥·∥\vec{y}∥) Equation (3)

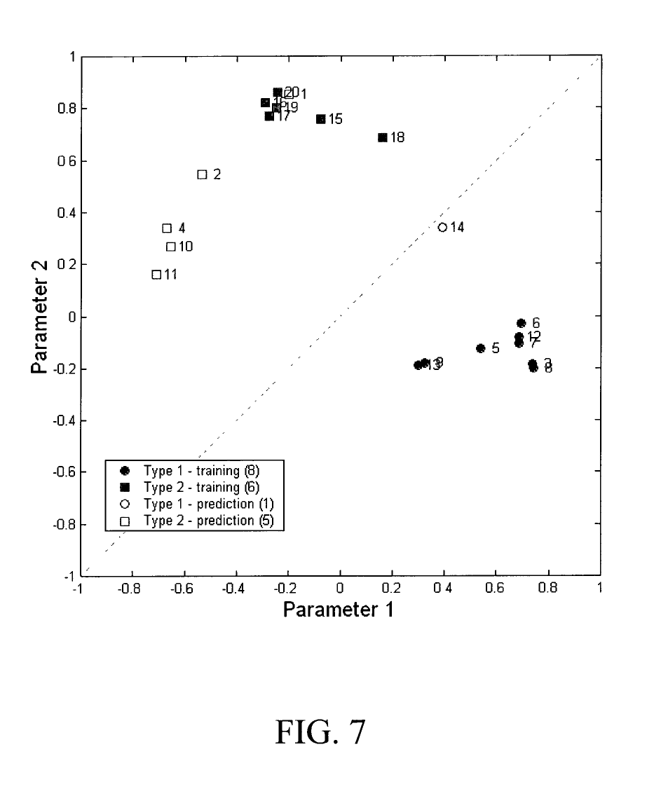

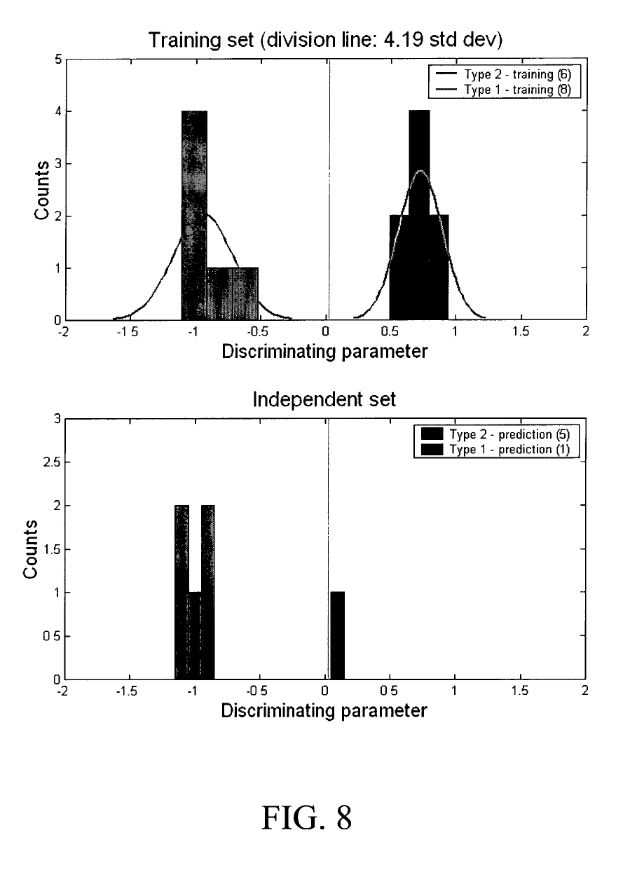

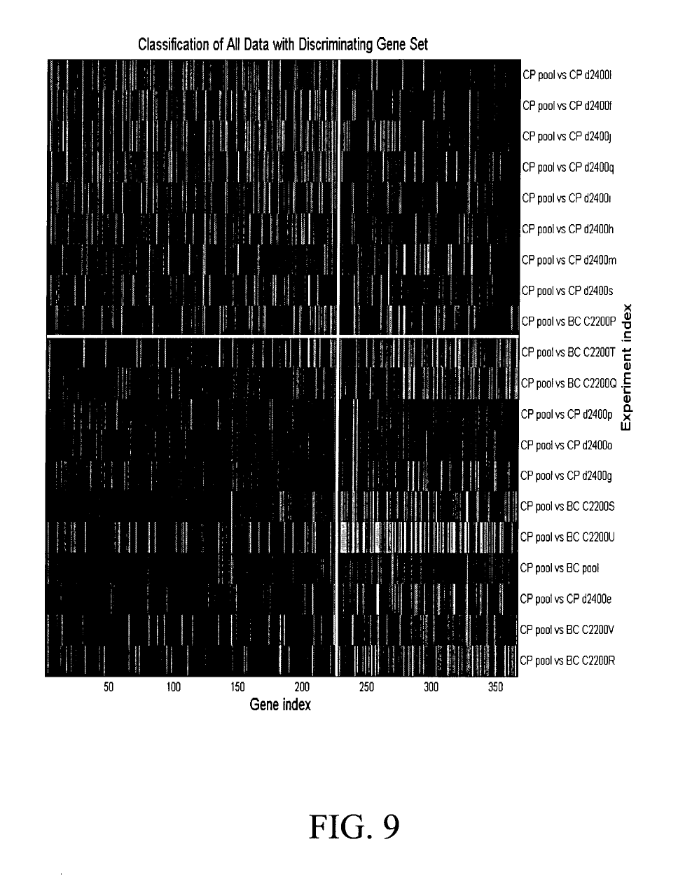

FIG. 7 shows the classification results of 20 experiments in the two-dimensional space of P1 and P2 based on the 366 reporter genes. In particular, a scatter plot of the correlation of each experiment with the CP template defined above and the correlation of each patient with the BC template defined above were shown. One can also reduce the two parameters into a single parameter as shown in FIG. 8. FIG. 9 shows expression patterns associated to the CML classification.

3. CML Progression Classification With Support Vector Machines

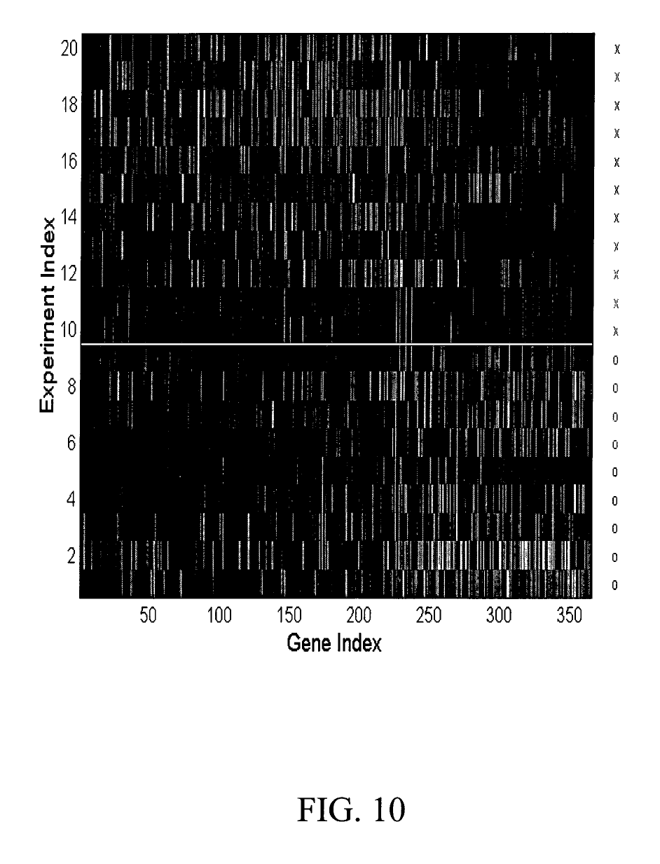

To test that the expression patterns found for the progression of CML patients are robust against the variation of methods and are reliable enough to apply to clinics, other supervised learning methods, such as a support vector machine, were applied to our data. FIG. 10 shows the classification results of 19 CML patients plus one CP pool vs BC pool profile obtained by applying support vector machine classifiers to the set of 366 genes.

### Example 3

### Construction of an Artificial Reference Pool

The reference pool for expression profiling in the above Examples was made by using equal amount of cRNAs from each individual patient in the sporadic group. In order to have a reliable, easy-to-made, and large amount of reference pool, a reference pool for CML diagnosis can be constructed using synthetic nucleic acid representing, or derived from, each marker gene. Expression of marker genes for individual patient sample is monitored only against the reference pool, not a pool derived from other patients.

To make the reference pool, 60-mer oligonucleotides are synthesized according to 60-mer ink-jet array probe sequence for each diagnostic/prognostic reporter genes, then double-stranded and cloned into pBluescript SK-vector (Stratagene, La Jolla, Calif.), adjacent to the T7 promoter sequence. Individual clones are isolated, and the sequences of their inserts are verified by DNA sequencing. To generate synthetic RNAs, clones are linearized with EcoRI and a T7 in vitro transcription (IVT) reaction is performed according to the MegaScript kit (Ambion, Austin, Tex.). IVT is followed by DNase treatment of the product. Synthetic RNAs are purified on RNeasy columns (Qiagen, Valencia, Calif.). These synthetic RNAs are transcribed, amplified, labeled, and mixed together to make the reference pool. The abundance of those synthetic RNAs are adjusted to approximate the abundance of the corresponding marker-derived transcripts in the real tumor pool.

### 2. REFERENCES CITED

All references cited herein are incorporated herein by reference in their entirety and for all purposes to the same extent as if each individual publication or patent or patent application was specifically and individually indicated to be incorporated by reference in its entirety for all purposes.

Many modifications and variations of the present invention can be made without departing from its spirit and scope, as will be apparent to those skilled in the art. The specific embodiments described herein are offered by way of example only, and the invention is to be limited only by the terms of the appended claims along with the full scope of equivalents to which such claims are entitled.

What is claimed is:

1. A method for classifying a cell sample as chronic phase CML (CP-CML) or blast crisis CML (BC-CML) comprising detecting a difference in the expression by said cell sample of a first plurality of genes relative to a control, said first plurality of genes consisting of at least 5 of the genes corresponding to the markers listed in Table 1.

2. The method of claim 1, wherein said plurality consists of at least 20 of the genes corresponding to the markers listed in Table 1.

3. The method of claim 1, wherein said plurality consists of at least 100 of the genes corresponding to the markers listed in Table 1.

4. The method of claim 1, wherein said plurality consists of at least 200 of the genes corresponding to the markers listed in Table 1.

5. The method of claim 1, wherein said plurality consists of each of the genes corresponding to the 366 markers listed in Table 1.

6. A method for classifying a sample as CP-CML or BC-CML by calculating the similarity between the expression of at least 20 of the markers listed in Table 1 in the sample to the expression of the same markers in a CP-CML nucleic acid pool and an BP-CML nucleic acid pool, comprising the steps of:
(a) labeling nucleic acids derived from a sample, with a first fluorophore to obtain a first pool of fluorophore-labeled nucleic acids;
(b) labeling with a second fluorophore a first pool of nucleic acids derived from two or more CP-CML samples, and a second pool of nucleic acids derived from two or more BP-CML samples:
(c) contacting said first fluorophore-labeled nucleic acid and said first pool of second fluorophore-labeled nucleic acid with said first microarray under conditions such that hybridization can occur, and contacting said first fluorophore-labeled nucleic acid and said second pool of second fluorophore-labeled nucleic acid with said second microarray under conditions such that hybridization can occur, detecting at each of a plurality of discrete loci on the first microarray a first flourescent emission signal from said first fluorophore-labeled nucleic acid and a second fluorescent emission signal from said first pool of second fluorophore-labeled genetic matter that is bound to said first microarray under said conditions, and detecting at each of the marker loci on said second microarray said first fluorescent emission signal from said first fluorophore-labeled nucleic acid and a third fluorescent emission signal from said second pool of second fluorophore-labeled nucleic acid;
(d) determining the similarity of the sample to the CP-CML and BP-CML pools by comparing said first fluorescence emission signals and said second fluorescence emission signals, and said first emission signals and said third fluorescence emission signals; and
(e) classifying the sample as CP-CML where the first fluorescence emission signals are more similar to said second fluorescence emission signals than to said third fluorescent emission signals, and classifying the sample as BC-CML where the first fluorescence emission signals are more similar to said third fluorescence emission signals than to said second fluorescent emission signals,
wherein said first microarray and said second microarray are similar to each other, exact replicas of each other, or are identical.

7. The method of claim 1, wherein said similarity is calculated by determining a first sum of the differences of expression levels for each marker between said first fluorophore-labeled nucleic acid and said first pool of second fluorophore-labeled nucleic acid, and a second sum of the differences of expression levels for each marker between said first fluorophore-labeled nucleic acid and said second pool of second fluorophore-labeled nucleic acid, wherein if said first sum is greater than said second sum, the sample is classified as CP-CML, and if said second sum is greater than said first sum, the sample is classified as BC-CML.

8. The method of claim 1, wherein said similarity is calculated by computing a first classifier parameter P₁ between an CP-CML template and the expression of said markers in said sample, and a second classifier parameter P₂ between an BC-CML template and the expression of said markers in said sample, wherein said P₁ and P₂ are calculated according to the formula:
Pᵢ=(\vec{z}ᵢ•\vec{y})/(∥\vec{z}ᵢ∥·∥\vec{y}∥),
wherein \vec{z}₁ and \vec{z}₂ are CP-CML and BC-CML templates, respectively, and are calculated by averaging said second fluorescence emission signal for each of said markers in said first pool of second fluorophore-labeled nucleic acid and said third fluorescence emission signal for each of said markers in said second pool of second fluorophore-labeled nucleic acid, respectively, and wherein \vec{y} is said first fluorescence emission signal of each of said markers in the sample to be classified as CP-CML or BC-CML, wherein the expression of the markers in the sample is similar to BC-CML if P₁<P₂, and similar to CP-CML if P₁>P₂.

9. A kit for determining the progression status of a sample, comprising at least two microarrays each comprising at least 20 of the markers listed in Table 1, and a computer system for determining the similarity of the level of nucleic acid derived from the markers listed in Table 1 in a sample to that in an CP-CML template and an BC-CML template, the computer system comprising a processor, and a memory encoding one or more programs coupled to the processor, wherein the one or more programs cause the processor to perform a method comprising computing the aggregate differences in expression of each marker between the sample and CP-CML pool and the aggregate differences in expression of each marker between the sample and BC-CML pool, or a method comprising determining the correlation of expression of the markers in the sample to the expression in the CP-CML and BC-CML pools, said correlation calculated according to Equation (3).

10. A microarray for distinguishing CP-CML from BC-CML cell samples comprising a positionally-addressable array of polynucleotide probes bound to a support, said polynucleotide probes comprising a plurality of different polynucleotide sequences, each of said nucleotide sequences comprising a sequence complementary and hybridizable to a different gene, said plurality consisting of at least 20 of the genes corresponding to the markers listed in Table 1.

11. A method for identifying the genes associated with a phenotype, comprising comparing the level of expression of a plurality of genes in a sample, the expression of which is correlated with the phenotype, to the level of expression of said plurality of genes in a first pool of nucleic acid derived from a plurality of samples, wherein said samples consist of normal individuals or individuals having a different phenotype than said sample.

12. The method of claim 11, wherein said sample is a second pool of nucleic acid, wherein said first pool and said second pool are derived from cell samples of individuals having different phenotypes.

13. The method of claim 13, wherein said first pool is derived from blast crisis CML samples, and said second pool is derived from chronic phase CML samples.

14. The method of claim “wherein said plurality of samples are from at least 2, 5, 10, 20 or 50 different individuals.

15. The method of claim 14 wherein each individual has cancer of a type selected from the group consisting of breast cancer, colon cancer, and prostate cancer.
